(19) United States
(12) Patent Application Publication
(10) Pub. No.: US 2019/0330142 A1
(43) Pub. Date: Oct. 31, 2019

(54) INHIBITORS OF MTOR-DEPTOR INTERACTIONS AND METHODS OF USE THEREOF

(71) Applicants: The Regents of the University of California, Oakland, CA (US); The United States Government represented by the Department of Veterans Affairs, Washington, DC (US)

(72) Inventors: Alan Lichtenstein, Los Angeles, CA (US); Michael E. Jung, Los Angeles, CA (US); Joseph F. Gera, Los Angeles, CA (US); Jihye Lee, Los Angeles, CA (US); Yijiang Shi, Santa Monica, CA (US)

(21) Appl. No.: 16/347,295

(22) PCT Filed: Nov. 6, 2017
(86) PCT No.: PCT/US17/60116

§ 371 Date: May 3, 2019

Related U.S. Application Data
(60) Provisional application No. 62/418,362, filed on Nov. 7, 2016.

(51) Int. Cl. C07C 251/84; C07C 281/04; A61P 35/00

(52) U.S. Cl. CPC C07C 251/84; C07C 2601/10; A61P 35/00; C07C 281/04

(57) ABSTRACT

Provided herein are substituted hydrazone compounds useful as inhibitors of DEPTOR. The invention further provides pharmaceutical compositions of the compounds of the invention. The invention also provides medical uses of substituted hydrazone compounds.

### RELATED APPLICATION

This application claims priority to U.S. Provisional Patent Application No. 62/418,362, filed on Nov. 7, 2016, which is hereby incorporated by reference in its entirety.

### GOVERNMENT INTEREST

This invention was made with Government support under R21 CA168491, awarded by the National Institutes of Health. The Government has certain rights in the invention. This work was supported by the U.S. Department of Veterans Affairs, and the Federal Government has certain rights in this invention.

### BACKGROUND

DEPTOR binds to mTOR and inhibits this kinase within TORC1 and TORC2 complexes. As an inhibitor of mTOR, it is not surprising that DEPTOR's expression is quite low in most tumor types. However, over-expression of DEPTOR occurs in the cancer cells from patients with multiple myeloma (MM).

Cells with the highest levels of DEPTOR over-expression are found in the specific genetic categories of MM that contain translocations between the IgH and MAF genes or copy number gains at chromosome 8q24² (a region that contains the DEPTOR gene). DEPTOR knockdown in high DEPTOR-expressing MM cell lines induces growth arrest and apoptosis. Since DEPTOR is an mTOR inhibitor, the proximal molecular effect of DEPTOR knockdown is activation of mTORC1 and mTORC2 activity. The finding that TORC1 paralysis protects MM cells against DEPTOR knock-down indicates that DEPTOR binding to mTOR with resulting TORC1 inhibition contributes to MM viability and proliferation. The anti-MM effects of DEPTOR silencing and singular over-expression in MM suggest DEPTOR is a potential therapeutic target in this malignancy.

Therefore, there is a continuing need to discover and develop new compounds that inhibit DEPTOR and that may be useful therapeutics.

### SUMMARY OF INVENTION

In certain embodiments, the invention relates to compounds having the structure of Formula (I):

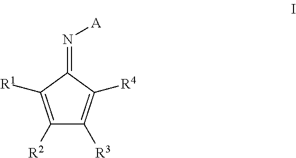

wherein
A is optionally substituted amino, alkylamino, cycloalkylamino, heterocyclylamino, arylamino, heteroarylamino, acylamino, diacylamino, or

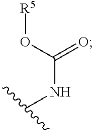

R¹, R², R³, and R⁴ are each, independently for each occurrence, H, halo or optionally substituted alkyl; and
R⁵ is, independently for each occurrence, H or optionally substituted alkyl, preferably branched alkyl, most preferably t-butyl.

In certain embodiments, R¹ is halo, e.g., Cl. In certain embodiments, R² is halo, e.g., Cl. In certain embodiments, R³ is halo, e.g., Cl. In certain embodiments, R⁴ is halo, e.g., CL.

In some embodiments, R¹, R², R³, and R⁴ are each halo, preferably each F or Cl, most preferably Cl.

In certain embodiments, A is —NHR⁶ or —NR⁶R⁷ (preferably —NHR⁶); R⁶ and R⁷ are each, independently for each occurrence, optionally substituted alkyl, optionally substituted cycloalkyl, optionally substituted aryl (e.g., phenyl), or optionally substituted heteroaryl; preferably optionally substituted alkyl or optionally substituted aryl (e.g., optionally substituted phenyl). Where R⁶ or R⁷ is substituted phenyl, the substituents are preferably located at the meta- and para-positions of the ring. Thus, in certain preferred such embodiments, R⁶ is

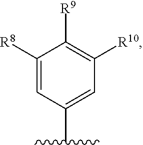

wherein R⁸, R⁹, and R¹⁰ are each, independently for each occurrence, H, optionally substituted alkyl, optionally substituted alkenyl, optionally substituted alkynyl, or an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl. In some embodiments, R⁸ and R⁹ are H and R¹⁰ is halo. In other embodiments, R⁹ is H and R⁸ and R¹⁰ are halo. In still other embodiments, R⁸ and R⁹ are H and R¹⁰ is optionally substituted lower alkyl, e.g., —CH₃ or —CF₃.

In certain embodiments, A is

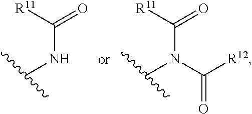

preferably

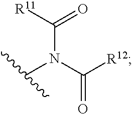

wherein
R¹¹ is optionally substituted alkyl or optionally substituted aryl or heteroaryl (e.g., optionally substituted phenyl); and
R¹² is optionally substituted aryl or heteroaryl (e.g., optionally substituted phenyl).

In certain embodiments, R¹¹ is phenyl, optionally substituted with an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl. In certain embodiments, R¹¹ is

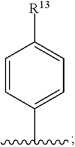

and R¹³ is, H, halo or optionally substituted alkyl. In some embodiments, R¹³ is F. In other embodiments, R¹³ is optionally substituted lower alkyl.

In certain embodiments, R¹² is phenyl, optionally substituted with an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl.

In certain preferred embodiments, R¹¹ and R¹² are the same.

In certain embodiments, R⁵ is optionally substituted lower alkyl.

The invention also relates to a pharmaceutical composition comprising a compound disclosed herein and pharmaceutically acceptable carrier.

The invention further relates to methods of treating cancer, inhibiting proliferation of a cancer cell, and inhibiting DEPTOR activity in a cell through the use of the compounds and composition disclosed herein. In certain embodiments, the cancer is breast cancer, prostate cancer, chronic myeloid leukemia, multiple myeloma, thyroid cancer, or lung cancer. In some embodiments, DEPTOR is over-expressed in the cancer cell. For example in certain embodiments DEPTOR is over-expressed in the cells of multiple myeloma.

### BRIEF DESCRIPTION OF THE FIGURES

FIG. 1 shows hit compounds from NCI inhibitor library identified as inhibitors of the DEPTOR-mTOR interaction.

FIG. 2 shows exemplary structural modifications of Compound B (NSC126405).

FIG. 3A-FIG. 3C depict exemplary assay data of compounds disclosed herein. FIG. 3A shows an immunoblot after 8226 cells exposed to drugs at 0.5 uM for 6 hrs, followed by immunoblot for expression of phosphorylated p70S6K, total p70 or actin. B-1=compound B from NCI; B-2=compound B synthesized at UCLA. FIG. 3B shows the summary of p70 phosphorylation data from 4 separate experiments (n=4) where derivatives were used at 0.5 uM, mean+SD; IC₅₀ from MTT cytotoxicity assays (n=4) shown below bars, mean+SD. FIG. 3C shows MTT cytotoxicity data (mean+/−SE, n=3) of four initially tested derivatives (4b, 3d, 3e and 3f).

FIG. 4A-FIG. 4E depict exemplary assay data of compounds disclosed herein. FIG. 4A shows representative experiment of p70 phosphorylation due to increasing concentrations of derivatives vs compound B (exposure is 6 hrs). FIG. 4B shows the summary of p70 phosphorylation data (mean+SD, n=4) shown as fold increase vs compound B (compound B arbitrarily kept at ‘1’) of densitometric ratio of phospho-p70/total p70 after exposure to increasing concentrations of derivatives for 6 hrs. FIG. 4C shows the upregulated p21 expression due to derivatives. FIG. 4D shows the MTT cytotoxicity assays of all derivatives vs compound B (48 hr assay), mean+SD, n=4. FIG. 4E shows % apoptosis (mean+SD, n=4) at 48 hrs after exposure to different derivatives.

FIG. 5A-FIG. 5E depict exemplary assay data of compounds disclosed herein. FIG. 5A shows the IC₅₀s of drug B and derivatives against 8226 MM cells or PBLs (48 hr assays, results are means of 5 separate experiments). Therapeutic indices (TIs) calculated as IC₅₀ PBLs/IC₅₀ for 8226 cells. FIG. 5B shows 8226 cells treated with DMSO or drug B (6 hrs) followed by immunoprecipitation of DEPTOR and precipitate then immunoblotted for DEPTOR or bound mTOR. FIG. 5C shows 8226 cells treated with DMSO or 0.5 uM of derivatives (6 hrs) followed by similar co-immunoprecipitation assay. FIG. 5D shows 8226 cells infected with lentivirus expressing either shRAPTOR or control shSCRAMBLE followed by immunoblot assay for RAPTOR, phosphorylated p70, total p70, DEPTOR or tubulin. FIG. 5E shows MM cells expressing either shSCRAMBLE or shRAPTOR incubated with increasing concentrations of derivatives, followed by MTT assay (48 hrs). Cytotoxicity (i.e., decreased cell survival) induced in RAPTOR-silenced cells was significantly reduced (p<0.05) compared to control shSCRAMBLE cells.

FIG. 6A-FIG. 6D depict exemplary assay data for compounds disclosed herein. Compound 3g demonstrated an enhanced therapeutic index versus drug NSC126405 when tested against myeloma cell lines 8226 (FIG. 6A), OPM2 (FIG. 6B) and H929 (FIG. 6C). FIG. 6D highlights IC₅₀ data for 3 g and Compound B (NSC126405). FIG. 6E shows % apoptosis at 48 hrs after exposure to 3 g and Compound B (NSC126405).

FIG. 7A-FIG. 7F depict exemplary assay data for compounds disclosed herein. FIG. 7A depicts data showing that compound 3g inhibits binding of DEPTOR to mTOR. FIG. 7B, FIG. 7C, and Fig. D depict data showing that compound 3g induces the rapid proteasome-dependent degradation of DEPTOR. FIG. 7E and FIG. 7F depict data showing the anti-tumor effect was blunted by further transfection of DEPTOR to over-express the protein.

FIG. 8A and FIG. 8B depict exemplary assay data for compound 3g showing that in a subcutaneous xenograft tumor model of myeloma growth, 3 g appears more efficacious than NSC 126405 (FIG. 8A) with only a minimal effect on normal WBC counts (FIG. 8B). Peripheral blood was analyzed for white blood cell (WBC), hematocrit (HCT), hemoglobin concentration (HgI) and platelet count.

### DETAILED DESCRIPTION OF THE INVENTION

In certain aspects, the invention provides substituted hydrazone compounds, and pharmaceutical compositions thereof. In particular, such substituted hydrazone compounds are useful as DEPTOR inhibitors, and thus can be used as anti-cancer agents.

### I. COMPOUNDS

In certain embodiments, the invention relates to compounds having the structure of Formula (I), or a pharmaceutically acceptable salt thereof:

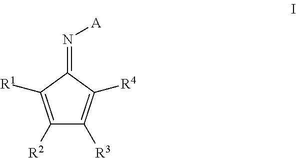

wherein
A is optionally substituted amino, alkylamino, cycloalkylamino, heterocyclylamino, arylamino, heteroarylamino, acylamino, diacylamino, or

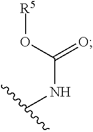

R¹, R², R³, and R⁴ are each, independently for each occurrence, H, halo or optionally substituted alkyl; and
R⁵ is, independently for each occurrence, H or optionally substituted alkyl, preferably branched alkyl, most preferably t-butyl.

In certain embodiments, R¹ is halo, e.g., Cl. In certain embodiments, R² is halo, e.g., Cl. In certain embodiments, R³ is halo, e.g., Cl. In certain embodiments, R⁴ is halo, e.g., CL. In some embodiments, R¹, R², R³, and R⁴ are each halo, preferably each F or Cl, most preferably Cl.

In certain embodiments, A is —NHR⁶ or —NR⁶R (preferably —NHR⁶);

R⁶ and R⁷ are each, independently for each occurrence, optionally substituted alkyl, optionally substituted cycloalkyl, optionally substituted aryl (e.g., phenyl), or optionally substituted heteroaryl; preferably optionally substituted alkyl or optionally substituted aryl (e.g., optionally substituted phenyl). Where R⁶ or R⁷ is substituted phenyl, the substituents are preferably located at the meta- and para-positions of the ring. Thus, in certain preferred such embodiments, R⁶ is

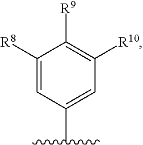

wherein R⁸, R⁹, and R¹⁰ are each, independently for each occurrence, H, optionally substituted alkyl, optionally substituted alkenyl, optionally substituted alkynyl, or an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl. In some embodiments, R⁸ and R⁹ are H and R¹⁰ is halo. In other embodiments, R⁹ is H and R⁸ and R¹⁰ are halo. In still other embodiments, R⁸ and R⁹ are H and R¹⁰ is optionally substituted lower alkyl, e.g., —CH₃ or —CF₃.

In certain embodiments, A is

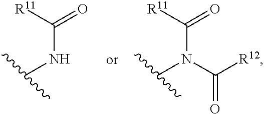

preferably

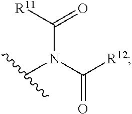

wherein
R¹¹ is optionally substituted alkyl or optionally substituted aryl or heteroaryl (e.g., optionally substituted phenyl); and
R¹² is optionally substituted aryl or heteroaryl (e.g., optionally substituted phenyl).

In certain embodiments, R¹¹ is phenyl, optionally substituted with an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl. In certain embodiments, R¹¹ is

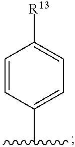

and R¹³ is, H, halo or optionally substituted alkyl. In some embodiments, R¹³ is F. In other embodiments, R¹³ is optionally substituted lower alkyl.

In certain embodiments, R¹² is phenyl, optionally substituted with an electron-withdrawing substituent (e.g., halogen, cyano, nitro, carbonyl, sulfonyl, etc.; that is, a substituent that does not have lone pairs that can electron-donate to the phenyl ring (such as an amino, hydroxy, alkoxy, etc.)), preferably H, halo or optionally substituted alkyl.

In certain preferred embodiments, R¹¹ and R¹² are the same.

In certain embodiments, R⁵ is optionally substituted lower alkyl.

In certain embodiments, A is

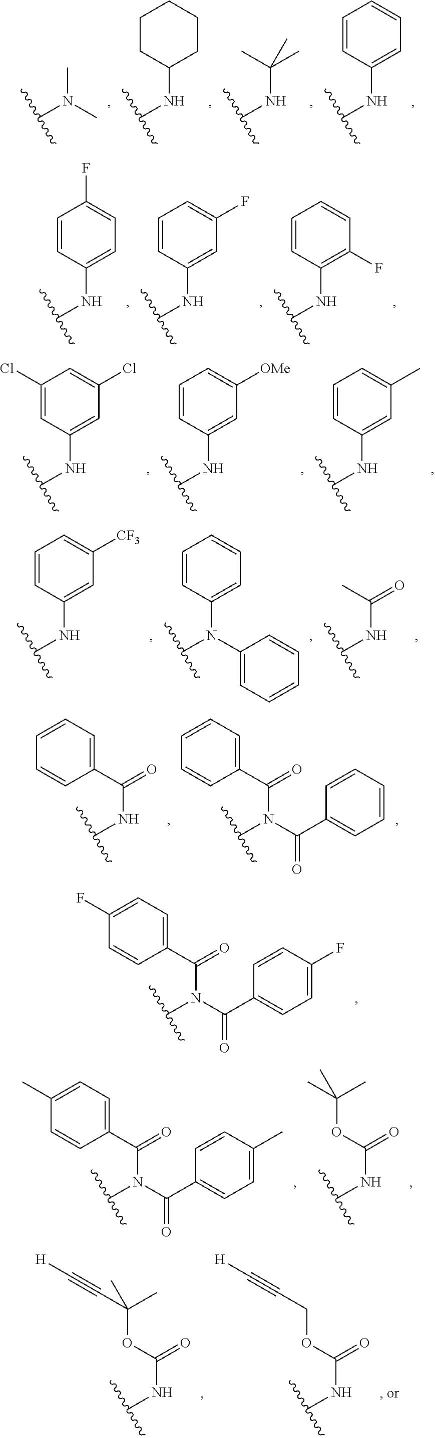

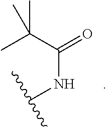 [SMILES: *NC(=O)C(C)(C)C]

In certain embodiments, compounds of the invention may be prodrugs of the compounds of Formula I, e.g., wherein a hydroxyl in the parent compound is presented as an ester or a carbonate, or carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. The compounds of the invention have more than one stereocenter. Consequently, compounds of the invention may be enriched in one or more diastereomer. For example, a compound of the invention may have greater than 30% de, 40% de, 50% de, 60% de, 70% de, 80% de, 90% de, or even 95% or greater de.

In certain embodiments, as will be described in detail below, the present invention relates to methods of treating or preventing cancer with a compound of Formula I, or a pharmaceutically acceptable salt thereof. In certain embodiments, the therapeutic preparation may be enriched to provide predominantly one enantiomer of a compound (e.g., of Formula I). An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound (e.g., of Formula I). A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient in the treatment of cancer, comprising an effective amount of any compound of Formula I, and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### II. PHARMACEUTICAL COMPOSITIONS

In certain embodiments, the present invention provides pharmaceutical compositions comprising a compound of Formula I and a pharmaceutically acceptable carrier.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of formula I) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### III. USES OF DEPTOR INHIBITORS

In certain aspects, the invention provides methods of treating cancer, comprising administering to a subject a compound of Formula I or a composition disclosed herein, e.g., in a therapeutically effective amount.

In certain embodiments, the cancer is breast cancer, prostate cancer, chronic myeloid leukemia, multiple myeloma, thyroid cancer, or lung cancer. In some embodiments, the cancer is multiple myeloma. In some embodiments, the cells of the multiple myeloma are characterized by overexpression of DEPTOR.

In certain embodiments, the invention provides methods of inhibiting proliferation of a cancerous cell comprising contacting a cancerous cell with an effective amount of a compound of Formula I. In some embodiments, DEPTOR is over-expressed in the cancer cell.

The invention also provides methods of inhibiting DEPTOR activity in a cell, comprising contacting a cell with a compound of Formula I or a composition of disclosed herein. In some embodiments, the cell overexpresses DEPTOR. In certain embodiments, the cell is a cancer cell. Such methods may be performed in vivo or in vitro.

In certain embodiments, the cancer is a solid tumor. The subject is generally one who has been diagnosed as having a cancerous tumor or one who has been previously treated for a cancerous tumor (e.g., where the tumor has been previously removed by surgery). The cancerous tumor may be a primary tumor and/or a secondary (e.g., metastatic) tumor.

In certain embodiments, the subject is a mammal, e.g., a human. In some embodiments, the subject has a high expression of DEPTOR in the cancerous cell.

### IV. DEFINITIONS

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, —OCF₃, ethoxy, propoxy, tert-butoxy and the like.

The term “cycloalkyloxy” refers to a cycloakyl group having an oxygen attached thereto.

The term “alkoxyalkyl” refers to an alkyl group substituted with an alkoxy group and may be represented by the general formula alkyl-O-alkyl.

The term “alkylaminoalkyl” refers to an alkyl group substituted with an alkylamino group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. Co alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

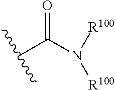

wherein each R¹⁰⁰ independently represent a hydrogen or hydrocarbyl group, or two R¹⁰⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

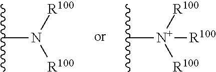

wherein each R¹⁰⁰ independently represents a hydrogen or a hydrocarbyl group, or two R¹⁰⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “arylamino” refers to an aryl or heteroaryl group, as defined herein, attached through an amino group.

The term “carbamate” is art-recognized and refers to a group

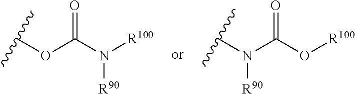

wherein R⁹⁰ and R¹⁰⁰ independently represent hydrogen or a hydrocarbyl group, such as an alkyl group, or R⁹⁰ and R¹⁰⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰⁰, wherein R¹⁰⁰ represents a hydrocarbyl group.

The term “carboxy”, as used herein, refers to a group represented by the formula —CO₂H.

The term “ester”, as used herein, refers to a group —C(O)OR¹⁰⁰ wherein R¹⁰⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The term “heteroalkylamino”, as used herein, refers to an amino group substituted with a heteralkyl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can also be substituted by oxo groups. For example, “heterocyclyl” encompasses both pyrrolidine and pyrrolidinone.

The term “heterocyclylamino”, as used herein, refers to an amino group substituted with a heterocyclyl group.

The term “heterocycloalkyl”, as used herein, refers to an alkyl group substituted with a heterocycle group.

The term “heterocycloalkylamino”, as used herein refers to an amino group substituted with a heterocycloalkyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, the term “oxo” refers to a carbonyl group. When an oxo substituent occurs on an otherwise saturated group, such as with an oxo-substituted cycloalkyl group (e.g., 3-oxo-cyclobutyl), the substituted group is still intended to be a saturated group. When a group is referred to as being substituted by an “oxo” group, this can mean that a carbonyl moiety (i.e., —C(═O)—) replaces a methylene unit (i.e., —CH₂—).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

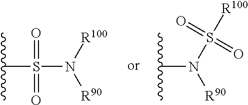

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰⁰, wherein R¹⁰⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰⁰, wherein R¹⁰⁰ represents a hydrocarbyl.

The term “thioalkyl”, as used herein, refers to an alkyl group substituted with a thiol group.

The term “thioester”, as used herein, refers to a group —C(O)SR¹⁰⁰ or —SC(O)R¹⁰⁰ wherein R¹⁰⁰ represents a hydrocarbyl.

The term “thioether”, as used herein, is equivalent to an ether, wherein the oxygen is replaced with a sulfur.

The term “urea” is art-recognized and may be represented by the general formula

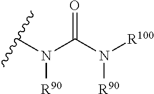

wherein R⁹⁰ and R¹⁰⁰ independently represent hydrogen or a hydrocarbyl, such as alkyl, or either occurrence of R⁹⁰ taken together with R¹⁰⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into the therapeutically active agents of the present invention (e.g., a compound of formula I). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of formula I in a formulation represented above can be replaced with the corresponding suitable prodrug, e.g., wherein a hydroxyl in the parent compound is presented as an ester or a carbonate or carboxylic acid present in the parent compound is presented as an ester.

### V. EXAMPLES

### Example 1. Chemistry

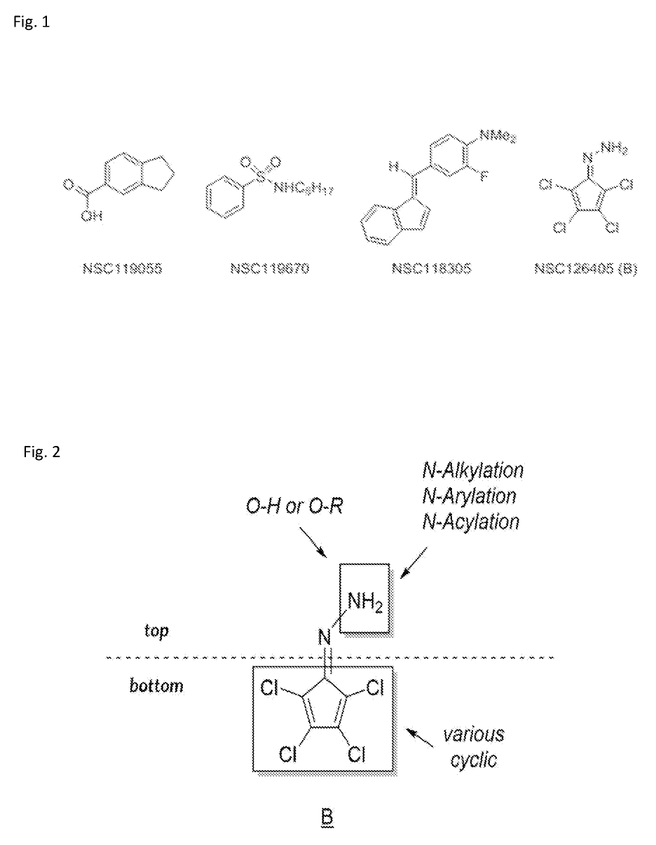

A pilot screening of ˜150,000 compounds from the NCI small molecule inhibitor library was conducted utilizing the yeast-2-hybrid screen, and identified four compounds (‘hits’) that specifically inhibited the DEPTOR-mTOR interaction (FIG. 1). Of these four, the first two compounds, NSC 119055 and NSC119670, did not offer many opportunities for structural variation since they are quite simple structures. The third compound, NSC118305, presented several positions for variation but we were worried about the conjugated diene unit since such polyolefinic units might give a rise to non-selective toxicity. Indeed, this compound was toxic to normal hematopoietic colony forming cells, completely preventing colony formation when used as low as 0.5 μM (not shown). The final compound NSC 126405 was not toxic to colony formation (in concentrations as high as 10 μM) and yet demonstrated molecular efficacy (enhanced mTORC1 activity) and anti-MM cytotoxicity (MTT assays). Therefore the final compound shown, NSC 126405, which was called simply compound B, was chosen as the first compound to modify to try to improve its activity.

The possible modifications of all parts of compound B are shown in FIG. 2. Since it has been reported that unsubstituted analogues of B, those with the chlorines removed, are quite reactive nucleophiles,⁵ likely due to the strong resonance structure which puts positive charge on the amino group and negative charge on the perchlorodiene system, a completely unsubstituted cyclopentadiene systems was not pursued, hoping that more substituted ones would be more stable and less reactive. A series of compounds were prepared and tested for their biological activity in order to establish a comprehensive structure-activity relationship (SAR) for this series.

First, the hydrazone unit was modified and in particular to vary the substituents on the hydrazone amine nitrogen, namely the top part of compound B as shown in FIG. 2. The synthesis of these compounds was accomplished by two relatively easy routes (Scheme 1).⁹ Thus condensation of commercially available hexachlorocyclopentadiene 1 with the selected hydrazine unit 2 in THF generally proceeded quite well. One could also use the HCl salts of the hydrazines and added base.⁶ The best procedure was often to use the hydrazine HCl salt in pyridine as solvent. The desired compounds 3 were normally purified by flash column chromatography on silica gel and several could be recrystallized as well. The parent compound B was prepared by this route in 62% yield. Several N-alkyl derivatives 3a-3c were prepared and also used this route to prepare some N-aryl derivatives 3d-3l by using either the alkyl hydrazines or the N-aminoanilines 2, where R¹ and/or R² was an aryl group. The compounds 3a-3l were generally quite deeply colored, e.g., dark orange or red.⁷

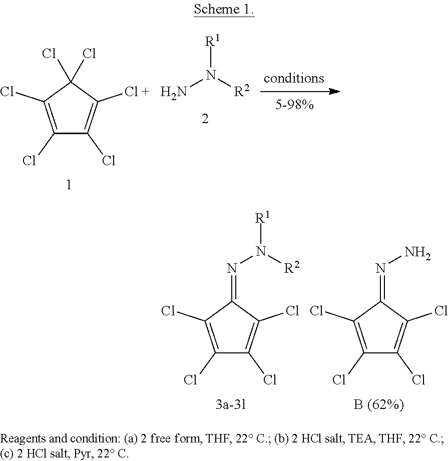

Next, several N-mono and di-acyl derivatives were prepared. See 4a-4f (Scheme 2).⁹ The monoacyl compounds 4a, 4c-4d were synthesized by selective mono-acylation of the parent compound B with either acid anhydrides or acyl chlorides in the presence of base as shown. If two equivalents of the acyl chloride were reacted with B and base, one obtained the diacylated derivatives 4b, 4e-4f. Also a few N-mono-carbamoyl derivatives 4 g-4i were prepared from B using di-tert-butyl dicarbonate or the corresponding alkyloxycarbonyl chloride.

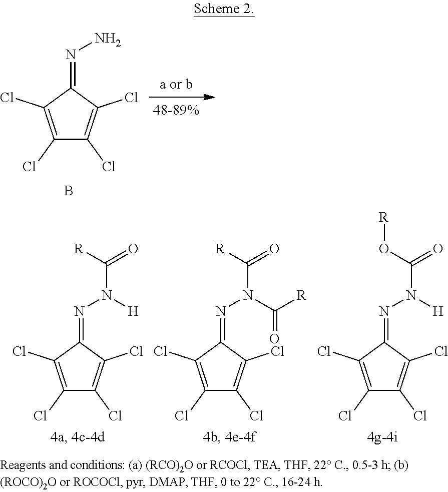

Since there was some concern that compounds with the dichloroalkene unit might show some non-specific toxicity, a few cyclic and acyclic moieties were introduced in place of the tetrachlorocyclopentadiene ring system (Scheme 3).^{10, 11} To expand our substrate scope further, modifications of the bottom part of the molecule were performed. The hydrazones 6a,¹⁰ 6b,¹¹ and 6d, were prepared by reaction of the simple ketones, fluorenone and xanthone, 5, with hydrazine or with hydrazine HCl salt and KOH in refluxing ethanol. The benzophenone hydrazine 6c was commercially available. The N-Boc derivative 6e was prepared from 6a by treating the hydrazine with di-tert-butyl dicarbonate and pyridine and DMAP in THF.¹² The yields of the hydrazones 6a-6e were generally quite good. Also, prepared were the unsubstituted hydrazones of indanone, cyclopentanone and acetophenone, but these compounds were unstable with respect to rearrangement to the dimeric azines.⁸

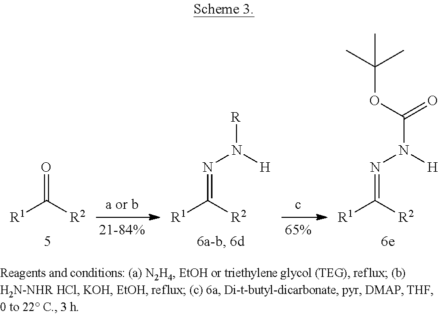

Finally, the oxime derivative 7a was prepared and the dimethoxy analogue 7b from hexachlorocyclopentadiene 1 (Scheme 4).¹³

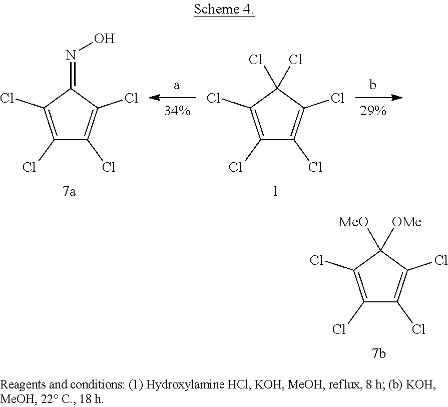 [SMILES: ON=C1C(Cl)=C(Cl)C(Cl)=C1Cl ; ClC1=C(Cl)C(Cl)(Cl)C(Cl)=C1Cl ; COC1(OC)C(Cl)=C(Cl)C(Cl)=C1Cl]

### Example 2: Chemical Syntheses

The general procedures used in the methods to prepare the compounds of the present invention are described below.

All reactions were carried out under open-air condition unless otherwise specified. Tetrahydrofuran (THF) was distilled from benzophenone ketyl radical under an argon atmosphere. Methanol, dichloromethane (DCM) and triethylamine (TEA) were distilled from calcium hydride under an argon atmosphere. Hexachlorocyclopentadiene was purchased from Chemieliva Pharmaceutical Co. in China and various hydrazines were purchased from Sigma-Aldrich, Alfa Aesar and TCI in ≥95% purity, all other solvents or reagents were purified according to literature procedures if necessary. ¹H-NMR spectra were recorded on Bruker spectrometers at 500 MHz and are reported relative to deuterated solvent signals (CHCl₃ δ 7.26; DMSO δ 2.48 ppm). Data for ¹H NMR spectra are reported as follows: chemical shift (δ ppm), multiplicity, coupling constant (Hz) and integration. Splitting patterns are designated as follows: s, singlet; d, doublet; t, triplet; q, quartet; dd, doublet of doublets; dt, doublet of triplets; td, triplet of doublets; tt, triplet of triplets; qd, quartet of doublets; qt, quartet of triplets; m, multiplet; and br, broad. ¹³C NMR spectra were recorded on Bruker Spectrometers at 125 MHz and are reported relative to deuterated solvent signals (CHCl₃ δ 77.0; DMSO δ 40.0 ppm). ¹⁹F NMR spectra were recorded on Bruker Spectrometers at 376.3 MHz and are reported relative to external Freon-113 in benzene (δ −73.75 ppm). Data for ¹³C and ¹⁹F NMR spectra are reported in terms of chemical shift. The chemical shifts are reported in parts per million (ppm, δ). Melting points were obtained using Buchi B-545 melting point apparatus and are uncorrected. The reactions were monitored with a silica gel TLC plate under UV light (254 and 365 nm) followed by visualization with a ninhydrin or phosphomolybdic acid staining solution. Column chromatography was performed on silica gel 60, 230-400 mesh. DART-HRMS spectra were collected on a Thermo Exactive Plus MSD (Thermo Scientific) equipped with an ID-CUBE ion source and a Vapur Interface (IonSense). Both the source and MSD were controlled by Excalibur, version 3.0. The purity of the compounds was assayed by high field proton and carbon NMR and was ≥95%.

### (Perchlorocyclopenta-2,4-dien-1-ylidene)hydrazine (B)

To a solution of hexachlorocyclopentadiene (1.6 mL, 10.0 mmol, 1.0 eq) in tetrahydrofuran (50 mL) was added hydrazine monohydrate (1.45 mL, 30.0 mmol, 3.0 eq) dropwise at 0° C. The reaction mixture was stirred for 10 min at room temperature and then concentrated in vacuo. The residue was purified by flash column chromatography over silica gel (hexane/ethyl acetate, 10:1, v/v) to afford the desired product B (1.44 g, 62%) as red brown solid: Rf=0.4 (hexane/ethyl acetate, 5:1, v/v); mp 187-189° C.; ¹H NMR (DMSO-d₆, 500 MHz) δ 10.67 (d, J=3.2 Hz, 1H), 9.93 (d, J=3.6 Hz, 1H); ¹H NMR (CDCl₃, 500 MHz) δ 8.09 (br, 2H); ¹³C NMR (DMSO-d₆, 125 MHz) δ 129.2, 125.6, 119.8, 118.2, 104.2; ¹³C NMR (CDCl₃, 125 MHz) δ 132.7, 131.2, 124.6, 119.5, 105.8 ppm; DART-HRMS found 230.88672 [M+H]⁺, calcd for C₅H₃Cl₄N₂ 230.90448.

### Representative Procedure for Synthesis of Alkyl and Aryl Hydrazones

### Method A. 1,1-Dimethyl-2-(perchlorocyclopenta-2,4-dien-1-ylidene)hydrazine (3a)

To a solution of hexachlorocyclopentadiene (0.16 mL, 1.0 mmol, 1.0 eq) in tetrahydrofuran (10 mL) was added unsym-dimethylhydrazine (0.23 mL, 3.0 mmol, 3.0 eq) dropwise at 0° C. The reaction mixture was stirred for 3 h at room temperature and then concentrated in vacuo. The residue was diluted with ethyl acetate (80 mL) and washed with water (2×20 mL) and brine (20 mL). The organic layer was dried with MgSO4, filtered and concentrated in vacuo. The residue was purified by flash column chromatography over silica gel (hexane/ethyl acetate, 10:1, v/v) to afford the desired product 3a (254 mg, 98%) as dark brown solid: Rf=0.45 (hexane/ethyl acetate, 3:1, v/v); mp 69-71° C.; ¹H NMR (CDCl₃, 500 MHz) δ 3.59 (s, 6H); ¹³C NMR (CDCl₃, 125 MHz) δ 129.5, 128.4, 121.8, 119.4, 103.0, 50.5 ppm; DART-HRMS found 258.93448 [M+H]⁺, calcd for C₇H₇C₁₄N₂ 258.93634.

### 1-(Perchlorocyclopenta-2,4-dien-1-ylidene)-2-phenylhydrazine (3d)

Dark brown solid (94% yield): Rf=0.65 (hexane/ethyl acetate, 3:1, v/v); mp 130-131° C.; ¹H NMR (CDCl₃, 500 MHz) δ 10.7 (s, 1H), 7.40 (td, J=7.5, 1.5 Hz, 2H), 7.34 (dd, J=9.0, 1.0 Hz, 2H), 7.15 (tt, J=7.5, 1.0 Hz, 1H); ¹³C NMR (CDCl₃, 125 MHz) δ 141.4, 130.94, 130.91, 129.7, 124.9, 123.9, 119.4, 115.1, 104.9 ppm; DART-HRMS found 306.91754 [M+H]⁺, calcd for C₁₁H₇Cl₄N₂ 306.93634.

### 1-(Perchlorocyclopenta-2,4-dien-1-ylidene)-2-(3-(trifluoromethyl)phenyl)hydrazine (3 g)

Red brown solid (46% yield): Rf=0.45 (hexane/ethyl acetate, 10:1, v/v); mp 146-148° C.; ¹H NMR (CDCl₃, 500 MHz) δ 10.69 (s, 1H), 7.53-7.52 (m, 3H), 7.38 (d, J=5.0 Hz, 1H); ¹³C NMR (CDCl₃, 125 MHz) δ 142.0, 132.2 (q, J_{CF}=32.0 Hz, 1C), 130.3, 126.9, 125.2, 124.8, 122.6, 121.1 (q, J_{CF}=3.5 Hz, 1C), 119.8, 117.9, 111.7 (q, J_{CF}=3.8 Hz, 1C), 105.4; ¹⁹F NMR (CDCl₃, 376 MHz, ¹H-dc) δ −62.90 ppm; DART-HRMS found 374.90492 [M+H]⁺, calcd for C₁₂H₆C₁₄F₃N₂ 374.92372.

### 1-(Perchlorocyclopenta-2,4-dien-1-ylidene)-2-(m-tolyl)hydrazine (3h)

Red brown solid (32% yield): Rf=0.65 (hexane/ethyl acetate, 5:1, v/v); mp 139-141° C.; ¹H NMR (DMSO-d₆, 500 MHz) δ 11.55 (s, 1H), 7.36 (s, 1H), 7.34 (d, J=9.0 Hz, 1H), 7.30 (t, J=7.5 Hz, 1H), 7.00 (d, J=7.5 Hz, 1H); ¹³C NMR (CDCl₃, 125 MHz) δ 141.4, 139.8, 130.8, 130.7, 129.5, 125.9, 123.7, 119.3, 115.6, 112.3, 104.8, 21.5 ppm; DART-HRMS found 320.93277 [M+H]⁺, calcd for C₁₂H₈Cl₄N₂ 320.95199.

### Method B. 1-Cyclohexyl-2-(perchlorocyclopenta-2,4-dien-1-ylidene)hydrazine (3b)

To a suspension of cyclohexylhydrazine HCl (527 mg, 3.5 mmol, 3.5 eq) in tetrahydrofuran (5 mL) was added triethylamine (0.49 mL, 3.5 mmol, 3.5 eq) and the mixture was stirred for 0.5 h. To a solution of hexachlorocyclopentadiene (0.16 mL, 1.0 mmol, 1.0 eq) in tetrahydrofuran (5 mL) was added the previously generated free form of cyclohexylhydrazine through filtration at room temperature. The reaction mixture was stirred for 12 h at room temperature and then concentrated in vacuo. The residue was diluted with ethyl acetate (80 mL) and washed with water (2×20 mL) and brine (20 mL). The organic layer was dried with MgSO4, filtered and concentrated in vacuo. The residue was purified by flash column chromatography over silica gel (hexane only) to afford the desired product 3b (60 mg, 19%) as red brown solid: Rf=0.5 (hexane only); mp 79-81° C.; ¹H NMR (DMSO-d₆, 500 MHz) δ 10.36 (d, J=4.0 Hz, 1H), 3.61-3.56 (m, 1H), 1.93-1.89 (m, 2H), 1.76-1.72 (m, 2H), 1.60-1.56 (m, 1H), 1.53 (qd, J=12.5, 3.5 Hz, 2H), 1.31 (qt, J=12.5, 3.5 Hz, 2H), 1.13 (qt, J=12.5, 3.5 Hz, 1H); ¹³C NMR (DMSO-d₆, 125 MHz) δ 127.3, 124.6, 118.7, 117.2, 103.5, 61.4, 31.6, 25.3, 24.7 ppm; DART-HRMS found 312.96460 [M+H]⁺, calcd for C₁₁H₁₃C₁₄N₂ 312.98329.

### 1-(tert-Butyl)-2-(perchlorocyclopenta-2,4-dien-1-ylidene)hydrazine (3c)

Red solid (5% yield): Rf=0.4 (hexane only); mp 80-82° C.; ¹H NMR (DMSO-d₆, 500 MHz) δ 9.98 (s, 1H), 1.34 (s, 9H); ¹³C NMR (CDCl₃, 125 MHz) δ 127.3, 125.1, 119.1, 117.3, 103.9, 59.2, 28.2 ppm; DART-HRMS found 286.96594 [M+H]⁺, calcd for C₉H₁₁Cl₄N₂ 286.96764.

### 1-(3,5-dichlorophenyl)-2-(perchlorocyclopenta-2,4-dien-1-ylidene)hydrazine (3f)

Brown solid (16% yield): Rf=0.6 (hexane/ethyl acetate, 5:1, v/v); mp 188-190° C.; ¹H NMR (CDCl₃, 500 MHz) δ 10.52 (s, 1H), 7.22 (d, J=2.0 Hz, 2H), 7.10 (t, J=2.0 Hz, 1H; ¹³C NMR (CDCl₃, 125 MHz) δ 143.3, 136.2, 133.0, 132.7, 125.7, 124.3, 119.9, 113.4, 105.6 ppm; DART-HRMS found 374.83957 [M+H]⁺, calcd for C₁₁H₅C₁₄N₂ 374.85839. Method C. 1-(3-Fluorophenyl)-2-(perchlorocyclopenta-2,4-dien-1-ylidene)hydrazine (3e). To a solution of hexachlorocyclopentadiene (0.16 mL, 1.0 mmol, 1.0 eq) in pyridine (5 mL) was added 3-fluorophenyl hydrazine HCl (244 mg, 1.5 mmol, 1.5 eq) at room temperature. The reaction mixture was stirred for 12 h at room temperature and then concentrated in vacuo. The residue was diluted with ethyl acetate (80 mL) and washed with water (2×20 mL) and brine (20 mL). The organic layer was dried with MgSO4, filtered and concentrated in vacuo. The residue was purified by flash column chromatography over silica gel (hexane only) to afford the desired product 3e (212 mg, 65%) as brown solid: Rf=0.6 (hexane/ethyl acetate, 5:1, v/v); mp 134-136° C.; ¹H NMR (CDCl₃, 500 MHz) δ 10.63 (s, 1H), 7.33 (dt, J=6.5, 8.5 Hz, 1H), 7.15 (dt, J=10.0, 2.0 Hz, 1H), 7.01 (dd, J=8.0, 1.5 Hz, 1H), 6.83 (td, J=8.0, 1.5 Hz, 1H); ¹³C NMR (CDCl₃, 125 MHz) δ 163.8 (d, J_{CF}=245.3 Hz, 1C), 143.2 (d, J_{CF}=10.4 Hz, 1C), 131.9, 131.7, 131.0 (d, J_{CF}=9.4 Hz, 1C), 124.8, 119.7, 1115. (d, J_{CF}=21.5 Hz, 1C), 110.7 (d, J_{CF}=2.9 Hz, 1C), 105.3, 102.3 (d, J_{CF}=26.8 Hz, 1C); ¹⁹F NMR (CDCl₃, 376 MHz, ¹H-dc) δ −110.42 ppm; DART-HRMS found 324.90814 [M+H]⁺, calcd for C₁₁H₆C₁₄FN₂ 324.92691.

### 1-(3-Methoxyphenyl)-2-(perchlorocyclopenta-2,4-dien-1-ylidene)hydrazine (3i)

Red brown solid (50% yield): Rf=0.4 (hexane/ethyl acetate, 10:1, v/v); mp 123-125° C.; ¹H NMR (CDCl₃, 500 MHz) δ 10.66 (s, 1H), 7.27 (t, J=8.0 Hz, 1H), 6.96 (s, 1H), 6.84 (dd, J=8.0, 1.0 Hz, 1H), 6.69 (dd, J=8.0, 1.5 Hz, 1H), 3.85 (s, 3H); ¹³C NMR (CDCl₃, 125 MHz) δ 161.0, 142.7, 131.0, 130.9, 130.5, 124.0, 119.4, 110.7, 107.7, 105.0, 100.6, 55.4 ppm; DART-HRMS found 336.92801 [M+H]⁺, calcd for C₁₂H₈Cl₄N₂O 336.94690.

### 1-(2-Fluorophenyl)-2-(perchlorocyclopenta-2,4-dien-1-ylidene)hydrazine (3j)

Red brown solid (52% yield): Rf=0.6 (hexane/ethyl acetate, 20:1, v/v); mp 120-122° C.; ¹H NMR (DMSO-d₆, 500 MHz) δ 11.16 (s, 1H), 7.65 (t, J=8.0 Hz, 1H), 7.36 (dd, J=10.0 Hz, 1H), 7.29 (t, J=7.5 Hz, 1H), 7.20 (dd, J=12.5, 6.0 Hz, 1H), 3.32 (s, 3H); ¹³C NMR (CDCl₃, 125 MHz) δ 151.1 (d, J_{CF}=242.4 Hz, 1C), 132.7, 131.8, 130.1, (d, J_{CF}=8.4 Hz, 1C), 125.3 (d, J_{CF}=3.5 Hz, 1C), 124.8, 124.6 (d, J_{CF}=7.3 Hz, IC), 119.5, 115.6, 115.5 (d, J_{CF}=17.4 Hz, 1C), 105.6; ¹⁹F NMR (CDCl₃, 376 MHz, ¹H-dc) δ −135.16 ppm; DART-HRMS found 324.90775 [M+H]⁺, calcd for C₁₁H₆C₁₄FN₂ 324.92691.

### 1-(4-Fluorophenyl)-2-(perchlorocyclopenta-2,4-dien-1-ylidene)hydrazine (3k)

Brown solid (48% yield): Rf=0.6 (hexane/ethyl acetate, 5:1, v/v); mp 147-149° C.; ¹H NMR (DMSO-d₆, 500 MHz) δ 11.64 (s, 1H), 7.59-7.56 (m, 2H), 7.30-7.26 (m, 2H); ¹³C NMR (CDCl₃, 125 MHz) δ 160.0 (d, J_{CF}=243.3 Hz, 1C), 137.8, 131.0, 130.9, 124.1, 119.4, 116.5 (d, J_{CF}=23.1 Hz, 1C), 116.4 (d, J_{CF}=7.9 Hz, 1C), 104.9; ¹⁹F NMR (CDCl₃, 376 MHz, ¹H-dc) δ −117.44 ppm; DART-HRMS found 324.90593 [M+H]⁺, calcd for C₁₁H₆C₁₄FN₂ 324.92691.

### 2-(Perchlorocyclopenta-2,4-dien-1-ylidene)-1,1-diphenylhydrazine (3l)

Dark red solid (78% yield): Rf=0.6 (hexane/ethyl acetate, 10:1, v/v); mp 128-130° C.; ¹H NMR (DMSO-d₆, 500 MHz) δ 7.48 (t, J=7.5 Hz, 4H), 7.37 (t, J=7.0 Hz, 2H), 7.34 (d, J=7.5 Hz, 4H); ¹³C NMR (DMSO-d₆, 125 MHz) δ 146.5, 132.2, 131.7, 130.4, 128.3, 123.6, 123.0, 121.7, 106.4 ppm; DART-HRMS found 381.95616 [M]⁺, calcd for C₁₇H₁₀Cl₄N₂ 381.95981.

### Representative Procedure for Synthesis of Mono and Diacyl Hydrazones

### Method A. N′-(Perchlorocyclopenta-2,4-dien-1-ylidene)benzohydrazide (4a)

To a solution of (perchlorocyclopenta-2,4-dien-1-ylidene)hydrazine (B, 116 mg, 0.5 mmol, 1.0 eq) in tetrahydrofuran (10 mL) was added benzoic anhydride (113 mg, 0.5 mmol, 1.0 eq) and triethylamine (0.07 mL, 0.5 mmol, 1.0 eq) dropwise in ice-bath. The reaction mixture was stirred for 3 h at room temperature and then concentrated in vacuo. The residue was diluted with ethyl acetate (150 mL) and washed with water (2×50 mL) and brine (50 mL). The organic layer was dried with MgSO4, filtered and concentrated in vacuo. The residue was purified by flash column chromatography over silica gel (hexane/ethyl acetate, 10:1, v/v) to afford the desired product 4a (239 mg, 71%) as brown solid: Rf=0.45 (hexane/ethyl acetate, 5:1, v/v); mp 170-172° C.; ¹H NMR (DMSO-d₆, 500 MHz) δ 11.98 (s, 1H), 7.93 (d, J=7.5 Hz, 2H), 7.69 (t, J=7.5 Hz, 1H), 7.59 (t, J=7.5 Hz, 2H); ¹³C NMR (DMSO-d₆, 125 MHz) δ 164.8, 140.8, 135.0, 133.7, 132.1, 129.5, 129.3, 128.7, 120.8, 109.7 ppm; DART-HRMS found 334.91239 [M+H]⁺, calcd for C₁₂H₇C₁₄N₂O 334.93125.

### N-Benzoyl-N′-(perchlorocyclopenta-2,4-dien-1-ylidene)benzohydrazide (4b)

Dark brown solid (52% yield): Rf=0.65 (hexane/ethyl acetate, 5:1, v/v); mp 123-124° C.; ¹H NMR (CDCl₃, 500 MHz) δ 7.15 (dd, J=8.5, 1.0 Hz, 2H), 8.02 (dd, J=8.5, 1.0 Hz, 2H), 7.68 (tt, J=7.5, 1.0 Hz, 1H), 7.56 (t, J=7.5 Hz, 1H), 7.53 (t, J=7.5 Hz, 2H), 7.48 (t, J=7.5 Hz, 2H); ¹³C NMR (CDCl₃, 125 MHz) δ 162.1, 151.0, 149.4, 137.7, 134.4, 132.6, 132.5, 130.6, 130.0, 128.9, 128.8, 128.1, 127.6, 120.2, 112.4 ppm; DART-HRMS found 438.93702 [M+H]⁺, calcd for C₁₉H₁₁C₁₄N₂O₂ 438.95747.

### N′-(Perchlorocyclopenta-2,4-dien-1-ylidene)acetohydrazide (4c)

Brown solid (89% yield): Rf=0.7 (hexane/ethyl acetate, 5:1, v/v); mp 145-147° C.; ¹H NMR (CDCl₃, 500 MHz) δ 10.62 (s, 1H), 2.42 (s, 3H); ¹³C NMR (CDCl₃, 125 MHz) δ 173.6, 136.3, 135.1, 129.1, 120.9, 107.5, 19.6 ppm; DART-HRMS found 272.89722 [M+H]⁺, calcd for C₇H₄C₁₄N₂O 272.91560.

### N′-(Perchlorocyclopenta-2,4-dien-1-ylidene)pivalohydrazide (4d)

Red brown solid (71% yield): Rf=0.5 (hexane/ethyl acetate, 5:1, v/v); mp 156-158° C.; ¹H NMR (DMSO-d₆, 500 MHz) δ 11.17 (s, 1H), 1.24 (s, 9H); ¹³C NMR (DMSO-d₆, 125 MHz) δ 175.4, 139.5, 134.5, 128.7, 120.7, 109.3, 39.3, 27.0 ppm; DART-HRMS found 314.94314 [M+H]⁺, calcd for C₁₀H₁₁Cl₄N₂O 314.96255.

### 4-Fluoro-N-(4-fluorobenzoyl)-N′-(perchlorocyclopenta-2,4-dien-1-ylidene)benzohydrazide (4e)

Brown solid (67% yield): Rf=0.75 (hexane/ethyl acetate, 5:1, v/v); mp 126-128° C.; ¹H NMR (CDCl₃, 500 MHz) δ 8.17 (dd, J=8.5, 5.0 Hz, 2H), 8.03 (dd, J=9.0, 5.0 Hz, 2H), 7.20 (t, J=9.0 Hz, 2H), 7.17 (t, J=8.5 Hz, 2H); ¹³C NMR (CDCl₃, 125 MHz) δ 166.6 (d, J_{CF}=255.4 Hz, 1C), 165.5 (d, J_{CF}=253.5 Hz, 1C), 161.1, 150.9, 149.9, 138.0, 133.4 (d, J_{CF}=9.6 Hz, 1C), 132.8, 130.6 (d, J_{CF}=9.0 Hz, 1C), 126.2 (d, J_{CF}=3.0 Hz, 1C), 123.7 (d, J_{CF}=2.9 Hz, 1C), 120.2, 116.3 (d, J_{CF}=9.3 Hz, 1C), 116.2 (d, J_{CF}=9.3 Hz, 1C), 112.3; ¹⁹F NMR (CDCl₃, 376 MHz, ¹H-dc) δ −102.42, −105.37 ppm; DART-HRMS found 474.91973 [M+H]⁺, calcd for C₁₉H₉C₁₄F₂N₂O₂ 474.93862.

### 4-Methyl-N-(4-methylbenzoyl)-N′-(perchlorocyclopenta-2,4-dien-1-ylidene)benzohydrazide (4f)

Brown solid (48% yield): Rf=0.75 (hexane/ethyl acetate, 5:1, v/v); mp 147-149° C.; ¹H NMR (CDCl₃, 500 MHz) δ 8.03 (d, J=8.0 Hz, 2H), 7.91 (d, J=8.0 Hz, 2H), 7.31 (d, J=8.0 Hz, 2H), 7.27 (d, J=10.5 Hz, 2H), 2.46 (s, 3H), 2.42 (s, 3H); ¹³C NMR (CDCl₃, 125 MHz) δ 162.2, 151.8, 149.4, 145.3, 143.3, 137.5, 132.3, 130.7, 129.7, 129.5, 128.2, 127.4, 127.9, 120.2, 112.3 ppm; DART-HRMS found 466.97013 [M+H]⁺, calcd for C₂₁H₁₅C₁₄N₂O₂ 466.98877.

### Method B. tert-Butyl 2-(perchlorocyclopenta-2,4-dien-1-ylidene)hydrazine-1-carboxylate (4 g)

To a solution of (perchlorocyclopenta-2,4-dien-1-ylidene)hydrazine (B, 116 mg, 0.5 mmol, 1.0 eq) in tetrahydrofuran (10 mL) was added pyridine (0.04 mL, 0.5 mmol, 1.0 eq) and 4-dimethylaminopyridine (12 mg, 0.1 mmol, 0.2 eq) at room temperature and the mixture was cooled down in ice bath. To the mixture, di-tert-butyl dicarbonate (164 mg, 0.75 mmol, 1.5 eq) in tetrahydrofuran (2 mL) was added dropwise at 0° C. The ice bath was removed and the reaction mixture was allowed to warm to room temperature and stirred for 16 h at room temperature and then concentrated in vacuo. The residue was diluted with ethyl acetate (100 mL) and washed with water (2×30 mL) and brine (30 mL). The organic layer was dried with MgSO4, filtered and concentrated in vacuo. The residue was purified by flash column chromatography over silica gel (hexane/ethyl acetate, 20:1, v/v) to afford the desired product 4h (90 mg, 54%) as orange solid: Rf=0.5 (hexane/ethyl acetate, 5:1, v/v); mp 108-110° C.; ¹H NMR (CDCl₃, 500 MHz) δ 10.22 (s, 1H), 1.56 (s, 9H); ¹³C NMR (CDCl₃, 125 MHz) δ 150.5, 135.9, 135.7, 128.2, 121.4, 107.2, 84.2, 28.0 ppm; DART-HRMS found 330.95319 [M+H]⁺, calcd for C₁₀H₁₁C₁₄N₂O₂ 330.95747.

### Prop-2-yn-1-yl 2-(perchlorocyclopenta-2,4-dien-1-ylidene)hydrazine-1-carboxylate (4h)

Red solid (19% and 30% RSM): Rf=0.5 (hexane/ethyl acetate, 5:1, v/v); mp 170-172° C.; ¹H NMR (CDCl₃, 500 MHz) δ 10.37 (s, 1H), 4.91 (d, J=2.5 Hz, 2H), 2.58 (t, J=2.5 Hz, 1H); ¹³C NMR (CDCl₃, 125 MHz) δ 151.3, 137.4, 136.9, 129.2, 121.5, 107.4, 76.5, 76.3, 54.6 ppm; DART-HRMS found 312.90698 [M+H]⁺, calcd for C₉H₅C₁₄N₂O₂ 312.91052

### 2-Methylbut-3-yn-2-yl 2-(perchlorocyclopenta-2,4-dien-1-ylidene)hydrazine-1-carboxylate (4i)

Red solid (50% yield): Rf=0.5 (hexane/ethyl acetate, 5:1, v/v); mp 143-145° C.; ¹H NMR (CDCl₃, 500 MHz) δ 10.27 (s, 1H), 2.62 (s, 1H), 1.80 (s, 6H); ¹³C NMR (CDCl₃, 125 MHz) δ 149.9, 136.6, 136.2, 128.6, 121.5, 107.3, 83.5, 75.2, 73.6, 28.9 ppm; DART-HRMS found 340.98938 [M+H]⁺, calcd for C₁₁H₉C₁₄N₂O₂ 340.94182.

### (9H-Fluoren-9-ylidene)hydrazine (6a)

To an ethanol (10 mL) solution of fluoren-9-one (360 mg, 2.0 mmol, 1.0 eq) was added hydrazine monohydrate (0.29 mL, 6.0 mmol, 3.0 eq) dropwise at room temperature. The reaction mixture was refluxed for 6 h and then concentrated in vacuo. The residue was diluted with ethyl acetate (100 mL) and washed with water (2×30 mL) and brine (40 mL). The organic layer was dried with MgSO4, filtered and concentrated in vacuo. The residue was purified by flash column chromatography over silica gel (hexane/ethyl acetate, 10:1, v/v) to afford the desired product 6a (315 mg, 81%) as yellow solid: Rf=0.15 (hexane/ethyl acetate, 10:1, v/v); mp 152-154° C.; 1H NMR (CDCl₃, 500 MHz) δ 7.91 (d, J=7.5 Hz, 1H), 7.77 (d, J=7.5 Hz, 1H), 7.73 (d, J=7.0 Hz, 1H), 7.65 (d, J=7.5 Hz, 1H), 7.44 (t, J=7.5 Hz, 1H), 7.37-7.29 (d, 3H), 6.41 (s, 2H); ¹³C NMR (CDCl₃, 125 MHz) δ 145.6, 141.3, 138.6, 137.7, 130.2, 129.7, 128.5, 127.9, 127.7, 125.5, 120.8, 120.5, 119.5 ppm; DART-HRMS found 195.09088 [M+H]⁺, calcd for C₁₃H₁₁N₂ 195.09222.

### (9H-Xanthen-9-ylidene)hydrazine (6b)

Yellow solid (21% yield): Rf=0.3 (hexane/ethyl acetate, 5:1, v/v); mp 126-128° C.; ¹H NMR (CDCl₃, 500 MHz) δ 8.32 (d, J=8.0 Hz, 1H), 7.91 (d, J=7.5 Hz, 1H), 7.43 (t, J=7.5 Hz, 1H), 7.34-7.30 (m, 2H), 7.21 (t, J=7.5 Hz, 1H), 7.18-7.15 (m, 2H), 5.80 (br, 2H); ¹³C NMR (CDCl₃, 125 MHz) δ 154.0, 151.8, 135.9, 130.8, 129.4, 127.4, 124.1, 123.9, 123.3, 122.6, 118.2, 117.5, 116.5 ppm; DART-HRMS found 211.08521 [M+H]⁺, calcd for C₁₃H₁₁N₂O 211.08714.

### 1-(9H-Fluoren-9-ylidene)-2-(3-fluorophenyl)hydrazine (6d)

To an ethanol (10 mL) suspension of 3-fluorophenylhydrazine HCl (325 mg, 2.0 mmol, 2.0 eq) was added triethylamine (0.29 mL. 2.1 mmol, 2.1 eq) and the mixture was stirred for 0.5 h. To the reaction mixture was added fluoren-9-one (180 mg, 1.0 mmol, 1.0 eq) and refluxed for 24 h. After the completion, the mixture was concentrated in vacuo. The residue was diluted with ethyl acetate (100 mL) and washed with water (2×30 mL) and brine (40 mL). The organic layer was dried with MgSO4, filtered and concentrated in vacuo. The residue was purified by flash column chromatography over silica gel (hexane/ethyl acetate, 10:1, v/v) to afford the desired product 6d (242 mg, 84%) as brown solid: Rf=0.4 (hexane/ethyl acetate, 5:1, v/v); mp 159-161° C.; ¹H NMR (CDCl₃, 500 MHz) δ 8.80 (s, 1H), 7.90-7.88 (m, 1H), 7.83 (d, J=8.0 Hz, 1H), 7.78 (d, J=7.5 Hz, 1H), 7.66-7.65 (m, 1H), 7.45 (td, J=7.5, 0.5 Hz, 1H), 7.39-7.27 (m, 4H), 7.18 (dt, J=11.0, 2.5, 1H), 6.98 (dd, J=8.0, 2.0 Hz, 1H), 6.68 (td, J=8.5, 2.5 Hz, 1H); ¹³C NMR (CDCl₃, 125 MHz) δ 164.0 (d, J_{CF}=242.8 Hz, 1C), 146.2 (d, J_{CF}=10.6 Hz, 1C), 141.5, 141.0, 138.1, 137.7, 130.5 (d, J_{CF}=9.8 Hz, 1C), 130.1, 129.8, 128.5, 128.0, 127.6, 124.4, 121.1, 120.9, 119.6, 109.3 (d, J_{CF}=2.5 Hz, 1C), 108.2 (d, J_{CF}=21.6 Hz, 1C), 101.0 (d, J_{CF}=26.5 Hz, 1C) ppm; DART-HRMS found 289.11254 [M+H]⁺, calcd for C₁₉H₁₄FN₂ 289.11355.

### tert-Butyl 2-(9H-fluoren-9-ylidene)hydrazine-1-carboxylate (6e)

To a solution of (9H-Fluoren-9-ylidene)hydrazine (6a, 97 mg, 0.5 mmol, 1.0 eq) in tetrahydrofuran (8 mL) was added pyridine (0.04 mL, 0.5 mmol, 1.0 eq) and DMAP (12 mg, 0.1 mmol, 0.2 eq) at room temperature, and the mixture was cooled with an ice-bath. To the mixture, di-tert-butyl dicarbonate (164 mg, 0.75 mmol, 1.5 eq) in tetrahydrofuran (2 mL) was added dropwise at 0° C. The ice bath was removed and the reaction mixture was allowed to warm to room temperature and stirred for 3 h at room temperature and then concentrated in vacuo. The residue was diluted with ethyl acetate (100 mL) and washed with water (2×30 mL) and brine (30 mL). The organic layer was dried with MgSO4, filtered and concentrated in vacuo. The residue was purified by flash column chromatography over silica gel (hexane/ethyl acetate, 10:1, v/v) to afford the desired product 6e (96 mg, 65%) as yellow solid: Rf=0.5 (hexane/ethyl acetate, 5:1, v/v); mp 106-107° C.; ¹H NMR (CDCl₃, 500 MHz) δ 8.85 (s, 1H), 7.93 (d, J=7.5 Hz, 1H), 7.79 (d, J=8.0 Hz, 1H), 7.74 (d, J=7.5 Hz, 1H), 7.61 (d, J=7.5 Hz, 1H), 7.47 (t, J=7.5 Hz, 1H), 7.38-7.35 (m, 2H), 7.31 (t, J=7.5 Hz, 1H), 1.61 (s, 9H); ¹³C NMR (CDCl₃, 125 MHz) δ 152.8, 146.1, 142.4, 139.1, 137.1, 130.9, 129.9, 129.8, 128.3, 127.9, 125.3, 122.3, 120.9, 119.5, 82.4, 28.3 ppm; DART-HRMS found 295.14353 [M+H]⁺, calcd for C₁₈H₁₉N₂O₂ 295.14353.

### 2,3,4,5-Tetrachlorocyclopenta-2,4-dien-1-one oxime (7a)

To hydroxylamine HCl (417 mg, 6.0 mmol, 6.0 eq) in 50 mL-round bottomed flask was added methanol (5 mL) and potassium hydroxide (337 mL, 6.0 mmol, 6.0 eq) in methanol (5 mL) at room temperature and the mixture was stirred for 1 h. Then the resultant KCl was filtered and the hydroxylamine solution was added to hexachlorocyclopentadiene (0.16 mL, 1.0 mmol, 1.0 eq) in methanol (5 mL) dropwise, and the mixture was refluxed for 6 h. After all hexachlorocyclopentadiene was consumed, the mixture was concentrated in vacuo. The residue was purified by flash column chromatography over silica gel (hexane/ethyl acetate, 10:1, v/v) to afford the desired product 7a (80 mg, 34%) as red brown solid: Rf=0.5 (hexane/ethyl acetate, 5:1, v/v); mp 178-180° C.; ¹H NMR (DMSO-d₆, 500 MHz) δ 14.52 (s, 1H); ¹³C NMR (DMSO-d₆, 125 MHz) δ 145.8, 134.2, 128.3, 119.0, 109.3 ppm; DART-HRMS found 231.87045 [M+H]⁺, calcd for C₅HCl₄NO 231.88905.

### 1,2,3,4-Tetrachloro-5,5-dimethoxycyclopenta-1,3-diene (7b)

To a methanol (5 mL) solution of hexachlorocyclopentadiene (0.48 mL, 3.0 mmol, 1.0 eq) was added potassium hydroxide (370 mL, 6.6 mmol, 2.2 eq) in methanol (5 mL) dropwise for 30 min at room temperature. The mixture was stirred for 18 h and the mixture was poured to chopped ice (70 mL). After the ice had melted, the mixture was extracted with dichloromethane (3×100 mL). The organic layer was dried with brine (100 mL) and MgSO4, filtered and concentrated in vacuo. The residue was purified by flash column chromatography over silica gel (hexane/ethyl acetate, 10:1, v/v) to afford the desired product 7b (230 mg, 29%) as light brown oil: Rf=0.15 (hexane/ethyl acetate, 10:1, v/v); ¹H NMR (CDCl₃, 500 MHz) δ 3.34 (s, 6H); ¹³C NMR (CDCl₃, 125 MHz) δ 129.4, 128.5, 104.8, 51.9 ppm.

### Example 3—Biological Results and Discussion

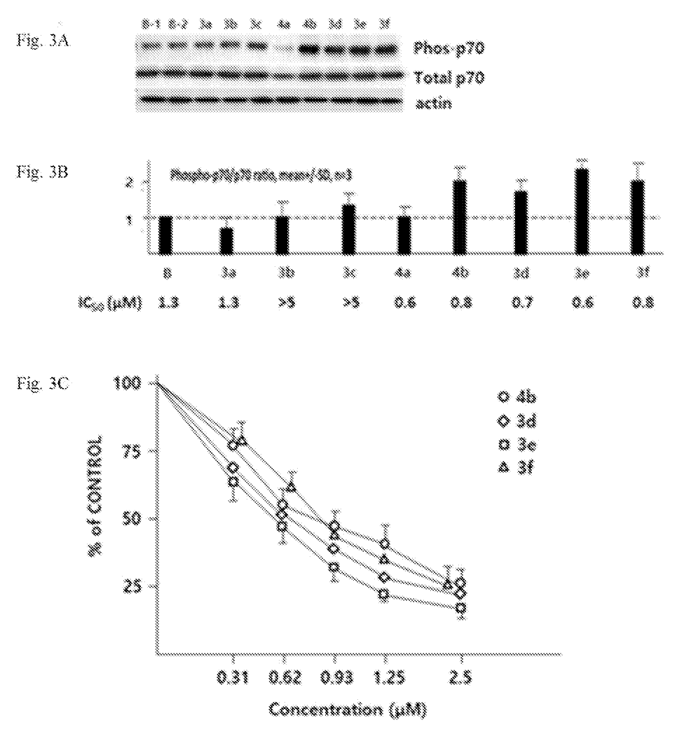

The first set of analogs, 3a-3f and 4a-4b, were compared against the parent compound B in assays for TORC1 activity. The 8226 cell line was used for these experiments as it demonstrates marked over-expression of DEPTOR.^{1,3} As drugs that prevent binding of the mTOR inhibitor, DEPTOR, to mTOR, effective drugs should increase mTOR kinase activity. In mTORC1, mTOR phosphorylates the p70S6 kinase. Thus, a Western blot was used to test for induction of p70 phosphorylation in this secondary screen. Compounds were tested at 0.5, 1, and 2 uM with 6 hrs in vitro exposure. Induction of p70 phosphorylation by all 8 derivatives was comparable to that of parent compound B when used at 1 or 2 uM except for the mono-benzoylated compound 4a which was toxic and showed degradation of p70. However, at 0.5 uM, the three N-aryl compounds 3d-3f and the dibenzoylated compound 4b were more effective than the parent compound B for induction of p70 phosphorylation (selected immunoblot shown in FIG. 3A). In contrast, the N-alkyl compounds 3a-3b did not exhibit a significant increase of activity compared to compound B, while the tert-butyl compound 3c showed only modest activity. Again, the mono-benzoylated compound 4a was toxic with considerable cell death seen even at this low concentration (0.5 uM). Follow-up experiments on compound 4a at lower concentrations which did not show toxicity (0.05-0.2 uM) demonstrated no enhancement of p70 phosphorylation suggesting compound 4a was non-specifically toxic. A summary of the p70 phosphorylation data from 4 separate experiments where derivatives were used at 0.5 uM, is shown in FIG. 3B. These first 8 derivatives were also screened for cytotoxicity against the same 8226 MM cell line in 48 hr MTT assays. The IC₅₀s for these assays are shown below the bar graphs in FIG. 3B and an example of one experiment is shown in FIG. 3C. In general, the analogs showed a correlation between the molecular effects (i.e., the ability to increase p70 phosphorylation) and their anti-MM cytotoxic effects. The four analogs (4b, 3d, 3e and 3f) with enhanced molecular effects compared to the parent compound B also demonstrated lower IC₅₀s. In contrast, compounds (3a-3c) which showed little or no enhancement of p70 phosphorylation compared to the parent compound B were also not enhanced for anti-MM cytotoxic. As mentioned above, compound 4a was cytotoxic without effects on p70 phosphorylation; thus, its anti-MM effects were assumed to be non-specific.

Based on the initial result from the first set of analogues 3a-3f and 4a-4b, further analogues in four categories were designed and synthesized, and evaluated their molecular and anti-MM cytotoxic activities in p70 phosphorylation and MTT assays. P70 phosphorylation was measured in 8226 cells treated for 6 hrs. Since the parent compound B was consistently ineffective in inducing p70 phosphorylation following exposure of 8226 cells to 0.5 uM, a 0.5 uM concentration was used to screen these additional derivatives for enhanced molecular activity. MTT (48 hr) assays exploited 8226 cells as well as an additional DEPTOR-over-expressing MM cell line, MM1.S. In general, MM1.S cells are less sensitive than 8226 to the cytotoxic activity of the parent compound B (IC₅₀ of 1.3 uM and 3.0 uM for 8226 and MM1.S, respectively). The structures and biological activity of all derivatives are shown in Tables 1-4, categorized by structural modification.

TABLE 1
p70 phosphorylation and cytotoxicity of alkyl and aryl analogues 3a-1

| | | | p70 | MTT | |
|---|---|---|---|---|---|
| | | | phosphorylation | (IC₅₀, uM) | |
| compd | R¹ | R² | vs the parent B^{a} | 8226 | MM1.S |
| B | H | H | 1.0 | 1.3 | 3.0 |
| 3a | CH₃ | CH₃ | no increase | >3 | >3 |
| 3b | C₆H₁₁ | H | no increase | >3 | >3 |
| 3c | C(CH₃)₃ | H | no increase | >3 | >3 |
| 3d | C₆H₅ | H | 1.8 | 0.6 | 2.0 |
| 3e | 3-FC₆H₄ | H | 2.2 | 0.5 | 1.5 |
| 3f | 3,5-Cl₂C₆H₃ | H | 1.9 | 0.8 | 2.0 |
| 3g | 3-CF₃C₆H₄ | H | 4.0 | 0.17 | 1.0 |
| 3h | 3-MeC₆H₄ | H | no increase | 2.0 | >3 |
| 3i | 3-MeOC₆H₄ | H | no increase | >3 | >3 |
| 3j | 2-FC₆H₄ | H | no increase | >3 | >3 |
| 3k | 4-FC₆H₄ | H | 5.0 | 0.7 | 1.3 |
| 3l | C₆H₅ | C₆H₅ | no increase | >3 | >3 |

^{a}fold increase, measured at 0.5 uM.

First, since the three N-arylated compounds 3d-3f showed promising results, aryl derivatives were prepared having either electron-withdrawing or electron-donating groups on the 3-position of phenyl ring 3 g-3i to examine the effect of phenyl substituents on the hydrazone in more detail. Table 1 shows p70 phosphorylation and MTT assay results of compound 3a-3l. The best activity was the 3-trifluoromethylphenyl analogue 3 g, namely a 4-fold increase in p70 phosphorylation and the IC₅₀ of 0.17 uM and 1.0 uM for the 8226 and MM1.S MM cell lines, respectively. The analogues having electron-donating group, 3h-3i, did not show good activity. Generally the analogues with the more electron-withdrawing substituents showed the best activity, e.g., 3 g and 3k. The effect of the position of the substituent was also examined. Although the 3-fluoro and 4-fluoro analogues, 3e and 3k, showed similar IC₅₀ values in the 8226 and MM1.S cell lines, curiously the 4-fluoro analogue 3k showed much better activity in the p70 phosphorylation assay, while the 2-fluoro analogue 3j showed no activity. Finally the diphenyl analogue 3l did not show any activity.

TABLE 2
p70 Phosphorylation and cytotoxicity of
acyl and carbamate derivatives 4a-4i

| | | | p70 | MTT | |
|---|---|---|---|---|---|
| | | | phosphorylation | (IC₅₀, uM) | |
| compd | | R | vs the parent B^{a} | 8226 | MM1.S |
| B | | | 1.0 | 1.3 | 3.0 |
| 4a | monoacyl | C₆H₅ | no increase | 0.6 | 0.8 |
| 4b | diacyl | C₆H₅ | 2.0 | 0.8 | 2.0 |
| 4c | monoacyl | CH₃ | no increase | 1.0 | ND |
| 4d | monoacyl | C(CH₃)₃ | 7.0 | 0.12 | 2.0 |
| 4e | diacyl | 4-FC₆H₄ | 5.0 | 0.25 | 0.3 |
| 4f | diacyl | 4-MeC₆H₄ | 2.5 | 0.5 | 0.5 |
| 4g | carbamate | C(CH₃)₃ | 6.0 | 0.1 | 0.6 |
| 4h | carbamate | CH₂C≡CH | 1.5 | 0.8 | ND |
| 4i | carbamate | C(CH₃)₂C≡CH | 1.2 | >5 | ND |

^{a}fold increase, measured at 0.5 uM; ND, not determined.

Next, a series of analogues of the mono- and di-acyl derivatives 4a-4f were examined as well as some carbamate derivatives 4 g-4i (Table 2). The presumed non-specific toxicity of the mono-benzoylated analogue 4a was again shown by the MTT assays with IC₅₀ values of 0.6 and 0.8 uM for 8226 and MM1.S, respectively. The mono-acetylated analogue 4c also showed no increase of p70 phosphorylation at 0.5 uM compared to compound B treatment, and similar cytotoxicity (vs drug B) to 8226 cells. On the other hand, the mono-pivaloylated analogue 4d showed a substantial 7-fold increase in p70 phosphorylation which correlated nicely with lowered IC₅₀ values for both cell lines in the MTT assays, 0.12 and 2.0 uM for 8226 and MM1.S, respectively.

Since the N-dibenzoylated compound 4b was quite promising in the initial testing, two additional dibenzoylated compounds were then prepared having different substituents on the 4-position of the phenyl ring. As expected, both compounds 4e and 4f showed good results for p70 phosphorylation and MTT assays. Moreover, in line with the results of the aryl derivatives, compound 4e having an electron-withdrawing substituent on the 4-position showed even better results than the other two diacyl derivatives. The carbamate derivatives 4 g-4i were also prepared and examined. The tert-butyloxycarbonyl analogue 4 g showed a substantial 6-fold increase in p70 phosphorylation and also enhanced cytotoxicity, 0.1 and 0.6 uM IC₅₀ for 8226 and MM1.S, respectively. The other carbamate derivatives 4h and 4i having a terminal acetylene were prepared for the possible further investigation of biotin protein labeling by click chemistry, but these compounds showed a minimal increase in mTORC1 activation. Only the simple propargyl compound 4h showed some cytotoxicity in the MTT assay.

It is perhaps interesting to compare the three analogues having a tert-butyl unit, namely the alkyl analogue, 3c, the acyl analogue 4d, and the carbamate 4 g. The alkyl analogue 3c showed the least activity in p70 phosphorylation, while the pivaloyl (tert-butylcarbonyl) analogue 4d and the t-Boc (tert-butyloxycarbonyl) analogue 4 g showed very good activities in both p70 phosphorylation and MTT assay.

TABLE 3
p70 Phosphorylation, cytotoxicity
and apoptosis of derivatives 6a-6e

| | | | | p70 | MTT | |
|---|---|---|---|---|---|---|
| | | | | phosphorylation | (IC₅₀, uM) | |
| compd | R¹ | R² | R | vs the parent B^{a} | 8226 | MM1.S |
| B | 2,3,4,5-Cl₄C₅ | | H | 1.0 | 1.5 | 3.0 |
| 6a | 9-Fluorenyl | | H | no increase | >5 | ND |
| 6b | 9-Xanthyl | | H | no increase | >5 | >5 |
| 6c | C₆H₅ | C₆H₅ | H | no increase | >5 | >5 |

| | | | | | |
|---|---|---|---|---|---|
| 6d | 9-Fluorenyl | 3-FC₆H₄ | no increase | >5 | >5 |
| 6e | 9-Fluorenyl | t-Boc | no increase | >5 | >5 |

^{a}fold increase, measured at 0.5 uM;
ND, not determined.

Next, the derivatives 6a-6e having cyclic and acyclic moieties other than the tetrachlorocyclopentadiene ring system were examined (Table 3). the unsubstituted hydrazine as the top unit was left unchanged and modified the bottom part to 9-fluorenyl 6a, 9-xanthyl 6b and benzophenone 6c. However, these new analogues did not show any improvement in both the p70 phosphorylation and the MTT assay. Since the 3-fluorophenyl (3e) and t-Boc carbamate (4 g) analogues showed enhanced biological activities with the tetrachlorocyclopentadiene as the bottom unit, these substituent on the 9-fluorenyl scaffold were introduced to give the analogues 6d and 6e, but these analogues also did not show any activity for reasons that are still unclear.

Finally, the oxime and dimethoxy derivatives, 7a and 7b, were examined and they both showed poor activity in the p70 phosphorylation and the MTT cytotoxicity assay (Table 4).

TABLE 4
p70 Phosphorylation, cytotoxicity and
apoptosis of derivatives 7a-7b

| | | p70 | | |
|---|---|---|---|---|
| | | phosphorylation | MTT | |
| | | vs the | (IC₅₀, uM) | |
| compd | structure | parent B^{a} | 8226 | MM1.S |
| B | hydrazone | 1.0 | 1.5 | 3.0 |
| 7a | oxime | no increase | >5 | ND |
| 7b | dimethoxy | no increase | >5 | ND |

^{a}fold increase, measured at 0.5 uM; ND, not determined.

All the derivatives in each category showed a rough correlation between the derivatives that were successful molecularly, namely able to increase p70 phosphorylation at 0.5 uM, and were effective cytotoxic compounds. Of the nine molecules (3d, 3e, 3f, 3 g, 3k, 4b, 4d, 4e and 4 g) active in the p70 phosphorylation assay (≥1.8× fold increase compared to compound B at 0.5 uM), all had enhanced cytotoxic effects (i.e., lower IC₅₀) compared to the parent B. Of the many compounds without molecular efficacy (i.e., <1.8× fold increase in p70 phosphorylation), only three—4a, 4f and 4h—demonstrated enhanced cytotoxic effects, which were presumably non-specific.

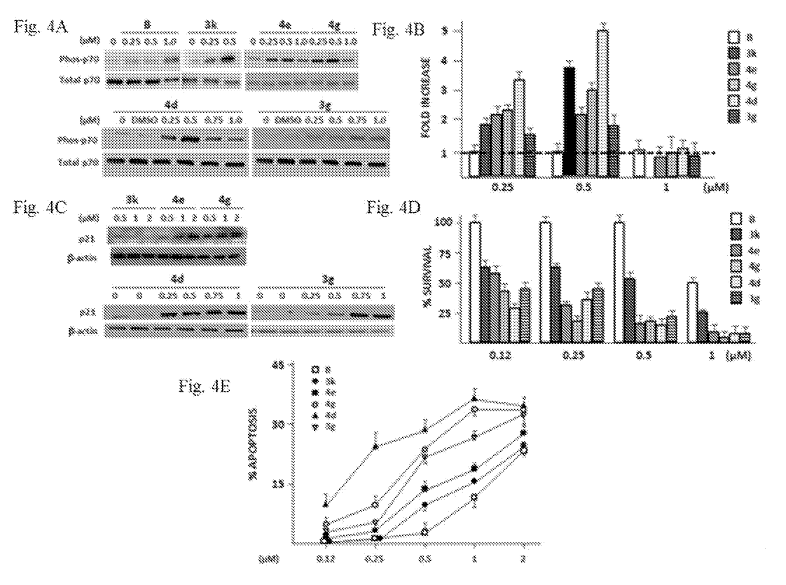

From these screening experiments, analogues 3 g, 3k, 4d, 4e and 4 g were identified as being the most active compounds in the p70 phosphorylation assay. These were then studied in more detail. As shown in FIGS. 4A and 4B, although inducing comparable amounts of p70 phosphorylation at 1 uM, when compared to parent compound B at lower concentrations, these biochemically modified compounds were significantly more effective as low as 0.25 uM. An additional molecular effect of either DEPTOR knockdown or parent compound B is an upregulation of p21 expression,^{3,4} believed to result from decreased TORC1-dependent expression of p21-targeting miRNAs.³ Upregulated expression of p21 contributes to the anti-MM cytotoxicity of DEPTOR targeting.³ As shown in FIG. 4C, some of these derivatives with enhanced TORC1 activation compared to parent compound B also demonstrated enhanced p21 expression, further strengthening the notion that their biochemical modifications allow more efficacious DEPTOR targeting. This was clearly shown for 4d, 4e, 4 g, and 3 g. FIG. 4D also demonstrates the enhanced anti-MM cytotoxicity of these agents in 8226 MTT assays. The ability of these drugs to enhance apoptosis in 8226 cells was tested and, as shown in FIG. 4E, their apoptosis activity was enhanced compared to parent compound B.

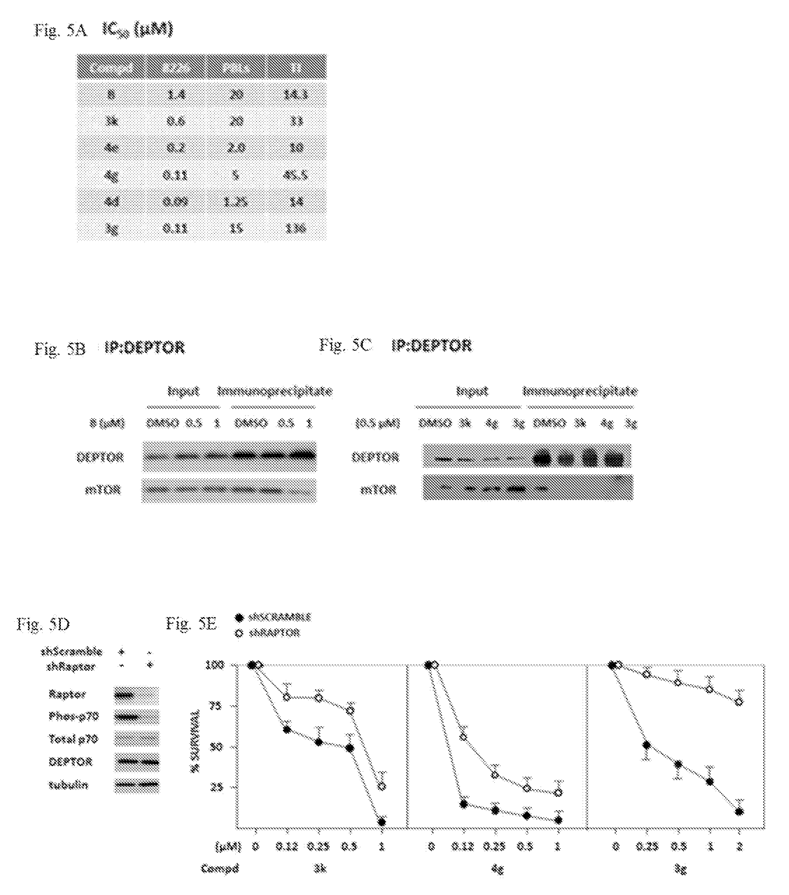

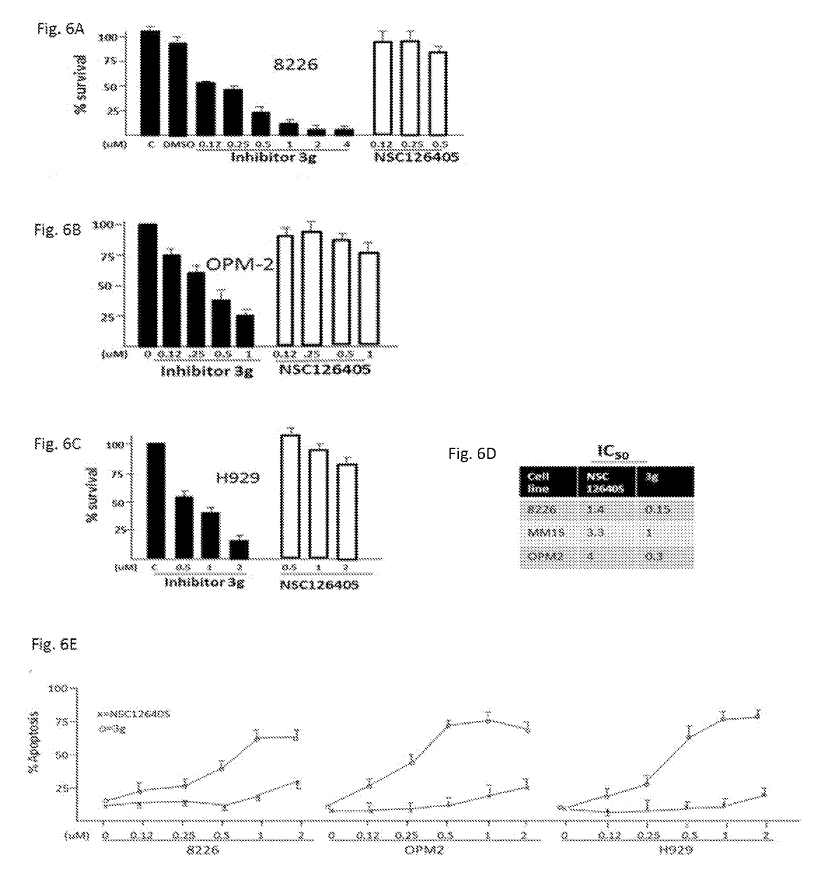

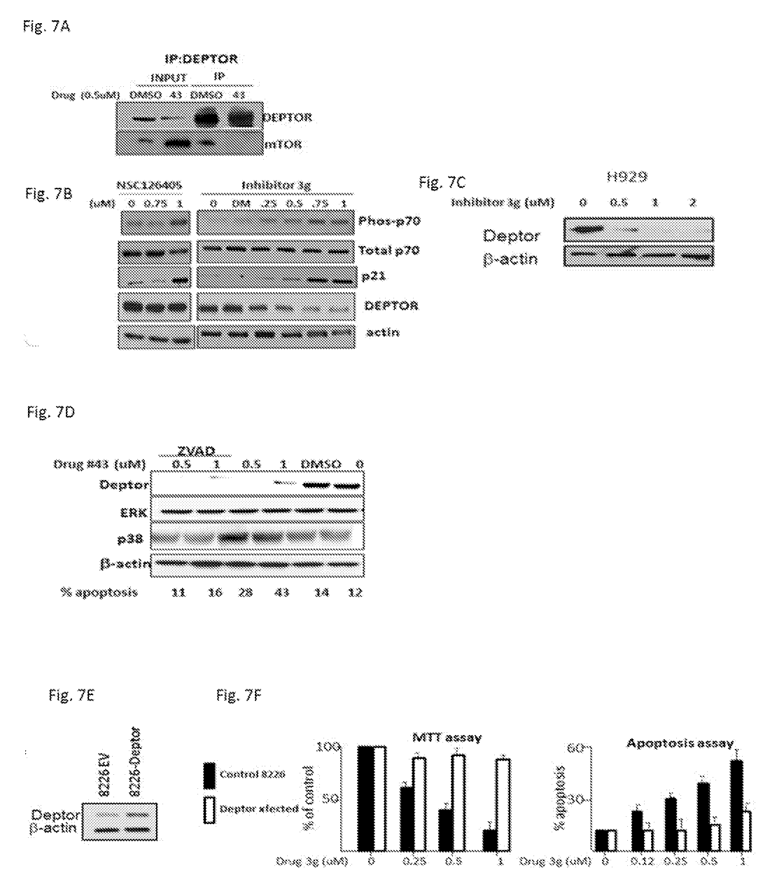

To compare anti-myeloma efficacy to non-specific toxicity, each of these 5 active derivatives were compared to compound B in their ability to inhibit survival of 8226 MM cells versus normal peripheral blood lymphocytes (PBLs). In head-to-head experiments, IC₅₀ values for each target were calculated and compared. As shown in FIG. 5A, although each of the derivatives demonstrated significantly reduced IC₅₀ values for the MM cells compared to compound B, they also showed variably enhanced toxicity to PBLs. However, three of the derivatives, 3 g, 3k and 4 g, showed significantly improved therapeutic indices (TIs) compared to parent compound B. To further support the fact that the ability of these three active derivatives (3 g, 3k and 4 g) to induce MM cell death was specifically related to their successful interference of DEPTOR/mTOR binding and mTORC1 activation, co-immunoprecipitation experiments were performed. Compound B prevents DEPTOR/mTOR binding in MM cells when used at 1 uM but 0.5 uM is ineffective (FIG. 5B). However, all three derivatives with enhanced TIs prevented DEPTOR/mTOR binding when used at 0.5 uM (FIG. 5C). These derivatives were also tested for their anti-MM cytotoxicity in isogenic lines with RAPTOR knockdown. FIG. 5D demonstrates the inhibitory effects of RAPTOR knockdown on mTORC1 activation. Finally as shown in FIG. 5E, MTT cytotoxicity assays also demonstrate a significantly decreased cytotoxicity induced by all three derivatives when tested against the RAPTOR-silenced MM cells providing some support that the molecular effects of these derivatives are linked to the cytotoxic effects.

### Example 4. Biological Assays

The following assay methods were used to identify and evaluate compounds of Formula (I) that are effective in inhibiting DEPTOR.

Cell Lines.

The 8226 and MM1.S myeloma cell lines were purchased from ATCC. The cell lines were characterized by FISH analysis and shown to contain MAF/Ig translocations. Western blot confirmed a significant over-expression of DEPTOR protein. Both lines were tested for mycoplasma within the last 6 months and were negative. Western blot assay—Protein was extracted and separated by 12.5% SDS-PAGE as previously described (see new ref below). Proteins were transferred to polyvinylidene difluoride membranes and their expression was detected utilizing specific antibodies purchased from Cell Signaling (Beverly, Mass.).

MTT Assay.

The MTT assay was performed by seeding 1-2×10⁴ target cells in 0.1 ml of complete media into wells of a 96 well microtiter plate. After incubation with compounds, the reduction of MTT to formazan by live cells was determined with a microplate ELISA reader equipped with a 570 nm filter. Quadruplicate wells were run for each group and the SD of each group was always <5% of the mean. Results are presented as % of control or % survival where OD of exp group was compared to the OD of a control group (cells incubated with DMSO alone) where the latter was arbitrarily made to be 100%.

Apoptosis Assay.

To identify apoptotic cells, a phycoerythrin (PE)-conjugated antibody specific for activated caspase 3 (BD Biosciences) was used. For staining, 10⁶ cells were washed with PB S and fixed and permeabilized with 0.5 ml cytofix/cytoperm solution. The cells were then incubated with a 1:5 dilution of PE-conjugated monoclonal anti-caspase 3 antibody for 30 mins and analyzed by flow cytometry.

RAPTOR Knockdown.

The short hairpin RNAs (shRNA)/pLKO.1, targeting RAPTOR or a scrambled sequence (control) were obtained from Addgene. Lentivirus was produced by the UCLA Vector Core facility and stable cell lines were made by transducing cells with lentivirus and selecting in geneticin.

Statistical Analysis.

The induction of p70 phosphorylation by derivative compounds was determined by densitometry, comparing immunoblot signals of phosphorylated p70 vs total p70. This ratio was then compared to that resulting from parental compound B, with the latter ratio arbitrarily placed at ‘1’. The IC₅₀ for MTT cytotoxicity was determined using a range of concentrations of derivatives. Percent apoptosis was enumerated by flow cytometry in drug-treated cultures by subtracting control apoptosis determined from DMSO-treated cultures. The percent apoptosis (i.e., positive staining for activated caspase 3) in the DMSO control cultures was always <15%.

### Example 5—Xenograft Tumor Model of Myeloma Growth

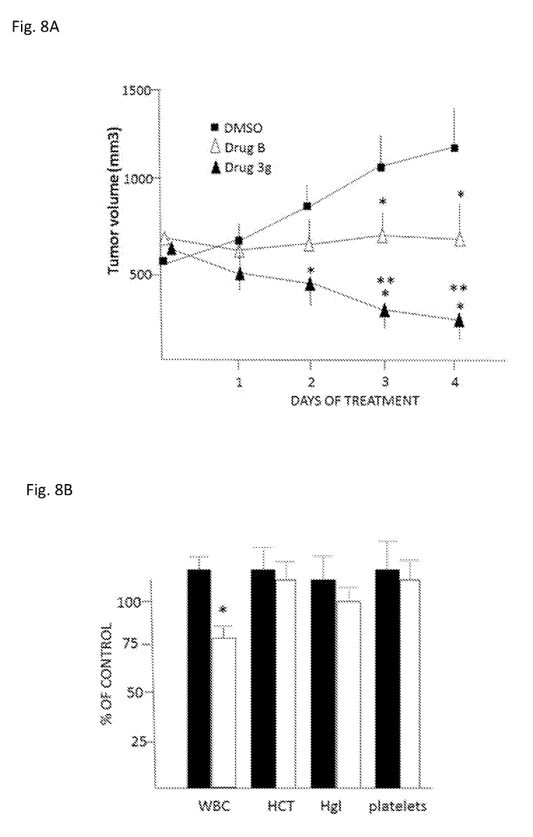

Briefly, mice were challenged subcutaneously with 5×10⁶ 8226 cells and when myeloma tumors reached 500 mm³, mice were treated with DMSO, compound B, or compound 3g (at 20 mg/kg) IP daily for 4 days. Tumor size (mean+/−SD, n=5) was assessed each day (FIG. 8A). *=different from control (DMSO), p<0.05, **=different from compound B, p<0.05. After 4 days of treatment, mice were sacrificed and peripheral blood analyzed for WBC, hematocrit (HCT), hemoglobin concentration (HgI) and platelet count (FIG. 8B). Data are % of control (determination in DMSO-Rx'd mice), mean+/−SD, n=5. *=different from DMSO control, p<0.05.

### REFERENCES

(1) Peterson, T. R.; Laplante, M.; Thoreen, C. C.; Sancak, Y; Kang, S. A.; Kuehl, W. M.; Gray, N. S.; Sabatini, D. M. DEPTOR is an mTOR inhibitor frequently overexpressed in multiple myeloma cells and required for their survival. Cell 2009, 137, 873-886.
(2) Carrasco, D. R.; Tonon, G.; Huang, Y; Zhang, Y; Sinha, R.; Feng, B. High resolution genomic profiles define distinct clinico-pathogenetic subgroups of multiple myeloma patients. Cancer Cell 2006, 9, 313-325.
(3) Yang, Y; Bardeleben, C.; Frost, P.; Hoang, B.; Shi, Y; Finn, R.; Gera, J.; Lichtenstein, A. DEPTOR is linked to a TORC1-p21 survival proliferation pathway in multiple myeloma cells. Genes & Cancer 2014, 5, 407-419.
(4) Shi, Y; Daniels-Wells, T.; Frost, P.; Lee, J.; Finn, R.; Bardeleben, C.; Penichet, M.; Jung, M. E.; Gera, J.; Lichtenstein, A. Cytotoxic properties of a DEPTOR-mTPR inhibitor in multiple myeloma cells. Cancer Res. 2016, 76, 5822-5831.
(5) Hafner, K.; Schulz, G.; Wagner, K. Cyclisch konjugierte 5-und 7-Ringsysteme I. 6-Amino-sowie 6-Hydroxy-fulvene und deren Aza-analoga. Liebigs Ann. Chem. 1964, 678, 39-53.
(6) Disselnkotter, H. Tetrachlor-diazo-cyclopentadien aus Hexachlor-cyclopentadien. Angew. Chem. 1964, 76, 431-432.
(7) For a discussion of the source of the color of these compounds and other analogues, see: Griffiths, J.; Lockwood, M. Chromogens based on Non-benzenoid Aromatic Systems. Part III. Synthesis, Spectra, and Molecular Orbital Calculations in the Substituted Fulvene and 6-Azafulvene Series. J. Chem. Soc. Perkin Trans 1 1976, 48-54.
(8) A discussion of this novel finding will be the subject of an upcoming manuscript.
(9) Dixon, D. D.; Ford, M. E. Preparation of N-Acyl substituted tetrachlorocyclopentadienone hydrazones. Synthesis 1980, 306-308.
(10) Bartucci, M. A.; Wierzbicki, P. M.; Gwengo, C.; Shajan, S. Hussain, S. H.; Ciszek, J. W. Synthesis of dihydroindolizines for potential photoinduced work function alteration. Tetrahedron Lett. 2010, 51, 6839-6842.
(11) Hafiz, M.; Taylor, G. A. Keten. Part 17. Addition reactions of ketens with N-phenyl nitrones. J. Chem. Soc., Perkin Trans. 1, 1980, 8, 1700-1705.
(12) Garcia-Ramos, Y; Proulx, C.; Lubell, W. D. Synthesis of hydrazine and azapeptide derivatives by alkylation of carbazates and semicarbazones. Can. J. Chem. 2012, 90, 985-993.
(13) Malihi, F.; Clive, D. L. J.; Chang, C.-C.; Minaruzzaman. Synthetic studies on CP-224,917 and CP-263,114: Access to advanced tetracyclic systems by intramolecular conjugate displacement and [2,3]-Wittig rearrangement. J. Org. Chem. 2013, 78, 996-1013.
(14) Tu, Y; Gardner A.; Lichtenstein, A. The phosphatidylinositol 3-kinase/AKT kinase pathway in multiple myeloma plasma cells: Roles in cytokine-dependent survival and proliferative responses. Cancer Res. 2000, 60, 6763-6770

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. The compound of claim 1, wherein the compound has a structure of Formula I 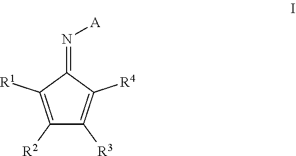
wherein
A is optionally substituted amino, alkylamino, cycloalkylamino, heterocyclylamino,
arylamino, heteroarylamino, acylamino, diacylamino, or 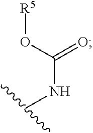
R¹, R², R³, and R⁴ are each, independently for each occurrence, H, halo or optionally substituted alkyl; and
R⁵ is, independently for each occurrence, H or optionally substituted alkyl.

2. The compound of any one of claim 1, wherein R¹ is halo.

3. The compound of claim 2, wherein R¹ is Cl.

4. The compound of any preceding claim, wherein R² is halo.

5. The compound of claim 4, wherein R² is Cl.

6. The compound of any preceding claim, wherein R³ is halo.

7. The compound of claim 6, wherein R³ is Cl.

8. The compound of any preceding claim, wherein R⁴ is halo.

9. The compound of claim 8, wherein R⁴ is Cl.

10. The compound of any preceding claim, wherein R¹, R², R³, and R⁴ are each halo, preferably each F or Cl.

11. The compound of any preceding claim, wherein A is —NHR⁶ or —NR⁶R⁷; R⁶ and R⁷ are each, independently for each occurrence, optionally substituted alkyl, optionally substituted cycloalkyl, optionally substituted aryl, or optionally substituted heteroaryl.

12. The compound of claim 11, wherein R⁶ and R⁷ are each, independently for each occurrence, optionally substituted alkyl or optionally substituted aryl.

13. The compound of claim 12, wherein R⁶ and R⁷ are each, independently for each occurrence, optionally substituted phenyl.

14. The compound of claim 13, wherein the substituents are preferably located at the meta- and para-positions of the ring.

15. The compound of claim 14, wherein R⁶ and R⁷ are each, independently for each occurrence, 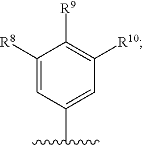
R⁸, R⁹, and R¹⁰ are each, independently for each occurrence, H, optionally substituted alkyl, optionally substituted alkenyl, optionally substituted alkynyl, or an electron-withdrawing substituent.

16. The compound of claim 15, wherein the electron-withdrawing substituent is a halogen or cyano, nitro, carbonyl, or sulfonyl group.

17. The compound of claim 15, wherein R⁸, R⁹, and R¹⁰ are each, independently for each occurrence, H, halo or optionally substituted alkyl.

18. The compound of claim 17, wherein R⁸ and R⁹ are H and R¹⁰ is halo.

19. The compound of claim 17, wherein R⁹ is H and R⁸ and R¹⁰ are halo.

20. The compound of claim 17, wherein R⁸ and R⁹ are H and R¹⁰ is optionally substituted lower alkyl.

21. The compound of claim 20, wherein R¹⁰ is —CH₃ or —CF₃.

22. The compound of any one of claims 1-10, wherein A is 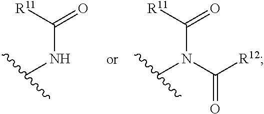
R¹¹ is optionally substituted alkyl or optionally substituted aryl or heteroaryl; and
R¹² is optionally substituted aryl or heteroaryl.

23. The compound of claim 22, wherein A is

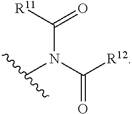

24. The compound of claim 22 or 23, wherein R¹¹ is optionally substituted alkyl or phenyl; and
R¹² is optionally substituted phenyl.

25. The compound of any one of claims 22-24, wherein the R¹¹ is phenyl, optionally substituted with an electron-withdrawing substituent.

26. The compound of claim 25, wherein the electron-withdrawing substituent is a halogen or cyano, nitro, carbonyl, or sulfonyl group.

27. The compound of any one of claims 22-24, wherein R¹¹ is 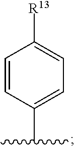
and R¹³ is H, halo or optionally substituted alkyl.

28. The compound of claim 27, wherein R¹³ is F.

29. The compound of claim 27, wherein R¹³ is optionally substituted lower alkyl.

30. The compound of any one claims 22-29, wherein R¹¹ and R¹² are the same.

31. The compound of any one of claims 1-10, wherein R⁵ is optionally substituted branched alkyl.

32. The compound of claim 31, wherein R⁵ is lower alkyl.

33. The compound of claims 31 or 32, wherein R⁵ is t-butyl.

34. The compound of any one of claims 1-10, wherein A is

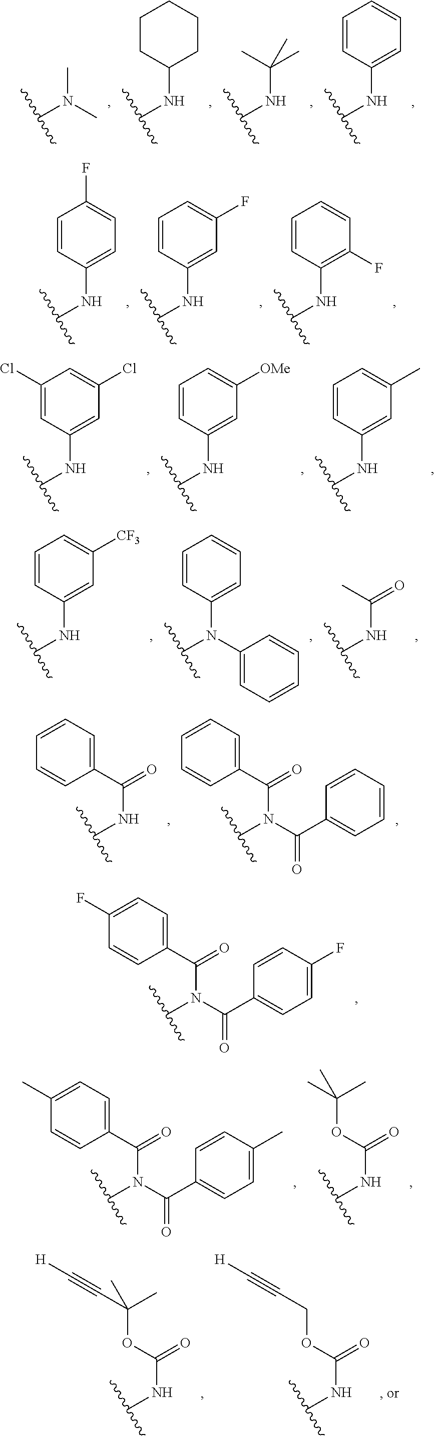

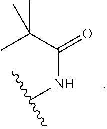 [SMILES: *NC(=O)C(C)(C)C]

35. The compound of claim 34, wherein A is

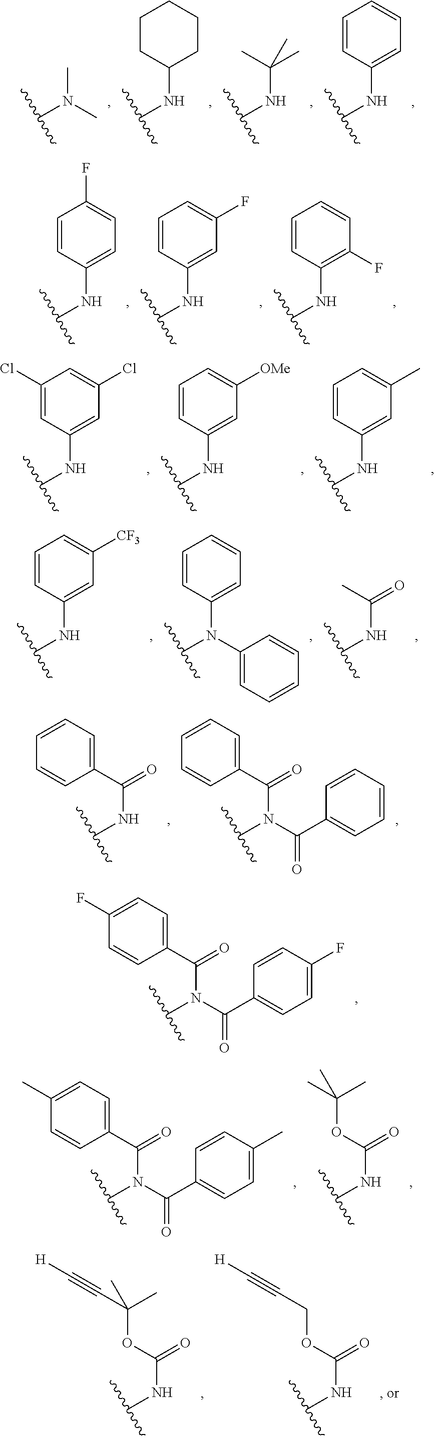

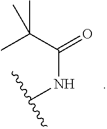 [SMILES: *NC(=O)C(C)(C)C]

36. A pharmaceutical composition comprising a compound of any preceding claim and pharmaceutically acceptable carrier.

37. A method of treating or preventing cancer in a subject, comprising administering to the subject a compound of any one of claims 1-35 or a composition of claim 36.

38. The method of claim 37, wherein the cancer is breast cancer, prostate cancer, chronic myeloid leukemia, multiple myeloma, thyroid cancer, or lung cancer.

39. The method of claim 38, wherein the cancer is multiple myeloma.

40. The method of claim 39, wherein cells of the multiple myeloma are characterized by overexpression of DEPTOR.

41. A method of inhibiting proliferation of a cancer cell, comprising contacting the cancer cell with a compound of any one of claims 1-35 or a composition of claim 36.

42. The method of claim 41, wherein DEPTOR is over-expressed in the cancer cell.

43. A method of inhibiting DEPTOR activity in a cell, comprising contacting the cell a compound of any one of claims 1-35 or a composition of claim 36.

44. The method of claim 43, wherein the cell overexpresses DEPTOR.
